(19) United States
(12) Patent Application Publication
(10) Pub. No.: US 2026/0363724 A1
(43) Pub. Date: Nov. 19, 2026

(54) DEVICES, SYSTEMS AND METHODS FOR TREATING THE SKIN

(71) Applicant: HydraFacial LLC, Long Beach, CA (US)

(72) Inventors: Joseph Wong, South Pasadena, CA (US); Stacie Depner, Anaheim, CA (US); Scott Garner, Stanton, CA (US); Ke Xu, Los Angeles, CA (US); Sheldon Phillips, Glendale, CA (US); Victor Yu, Vancouver (CA); Shirin Toloue, Port Coquitlam (CA); Aaron MacDonald, Garibaldi Highlands (CA); Malcolm Dimeglio, Vancouver (CA); Mark Mosher, North Vancouver (CA)

(21) Appl. No.: 19/138,594

(22) PCT Filed: Dec. 12, 2023
(86) PCT No.: PCT/US2023/083689

§ 371 Date: Jun. 12, 2025

Related U.S. Application Data
(60) Provisional application No. 63/432,691, filed on Dec. 14, 2022.
(60) Provisional application No. 63/470,429, filed on Jun. 1, 2023.

(51) Int. Cl. A61B 17/3203; A61B 17/00

(52) U.S. Cl. CPC A61B 17/3203; A61B 2017/00084; A61B 2017/00119; A61B 2017/00367; A61B 2017/00473; A61B 2017/00477; A61B 2017/00761; A61B 2217/005

(57) ABSTRACT

A skin treatment device may comprise a handpiece configured to receive a tip along its distal end. The device comprises a tip detection assembly configured to automatically detect a property of the tip secured to the handpiece, wherein the tip detection assembly comprises at least one light emitter and at least one light detector, wherein light emitted from the at least one light emitter is configured to be detected by the at least one light detector, a light path existing between the at least one light emitter and the at least one light detector.

### CROSS-REFERENCE TO RELATED APPLICATIONS

This application claims priority to U.S. Provisional Patent Application No. 63/432,691, filed Dec. 14, 2022, and U.S. Provisional Patent Application No. 63/470,429, filed Jun. 1, 2023, the entire contents of both of which are incorporated herein by reference in their entireties.

### BACKGROUND

### Field

This application relates generally to skin treatment, and more specifically, to apparatuses, systems and methods for treating a person's skin.

### Description of the Related Art

Abrasion of the outer layer or epidermis of the skin is desirable to smooth or blend scars, blemishes, or other skin conditions that may be caused by, for example, acne, sun exposure, and aging. Standard techniques used to abrade the skin have generally been separated into two fields referred to as dermabrasion and microdermabrasion. Both techniques remove portions of the epidermis called the stratum corneum, which the body interprets as a mild injury. The body then replaces the lost skin cells, resulting in a new outer layer of skin. Additionally, despite the mild edema and erythema associated with the procedures, the skin looks and feels smoother because of the new outer layer of skin.

### SUMMARY

According to some embodiments, a skin treatment device comprises a handpiece comprising a distal end, the handpiece configured to receive a tip along the distal end, and a tip detection assembly configured to automatically detect a property of the tip secured to the handpiece, wherein the tip detection assembly comprises at least one light emitter and at least one light detector, wherein light emitted from the at least one light emitter is configured to be detected by the at least one light detector, a light path existing between the at least one light emitter and the at least one light detector, and wherein at least a portion of the tip is configured to be within the light path of the tip detection assembly when the tip is secured to the handpiece.

According to some embodiments, the tip detection assembly is configured to detect whether the tip is or may be new. In some embodiments, the tip detection assembly is configured to detect whether the tip should be used in a skin treatment procedure. In one embodiment, the tip detection assembly is configured to detect the type of tip that is secured to the handpiece. In some embodiments, the type of tip is based, at least in part, on the color of the tip.

According to some embodiments, the tip detection assembly is configured to detect differences in opacity of the at least a portion of the tip that is configured to be within the light path of the tip detection assembly when the tip is secured to the handpiece.

According to some embodiments, wherein the tip detection assembly is configured to detect differences in color or another property associated with light of the at least a portion of the tip that is configured to be within the light path of the tip detection assembly when the tip is secured to the handpiece. In some embodiments, the color or another property determined the type of abrasive structure included on the tip.

According to some embodiments, the at least a portion of the tip configured to be within the light path of the tip detection assembly when the tip is secured to the handpiece comprises a stem of the tip.

According to some embodiments, the at least a portion of the tip configured to be within the light path of the tip detection assembly when the tip is secured to the handpiece comprises a dissolvable coating or layer. In some arrangements, the dissolvable coating or layer comprises an opacity greater than 0% (e.g., 5% to 100%). In some embodiments, the dissolvable coating or layer is provided along a fluid path of the tip. In one embodiment, the dissolvable coating or layer is configured to at least partially dissolve when fluid passes from the handpiece to the tip during use. In some embodiments, the tip detection assembly is configured to detect an opacity or other light property of the at least a portion of the tip configured to be within the light path of the tip detection system when the tip is secured to the handpiece. In some arrangements, the device is configured to terminate or not commence a skin treatment procedure if the opacity or other light property of the at least a portion of the tip configured to be within the light path of the tip detection system when the tip is secured to the handpiece is below a threshold level. In some embodiments, the device is configured to provide at least one alert if the opacity or other light property of the at least a portion of the tip configured to be within the light path of the tip detection system when the tip is secured to the handpiece is below a threshold level. In some embodiments, the at least one alert comprises at least one of an audible alert, a visual alert and a haptic alert.

According to some embodiments, the tip detection system comprises at least one light pipe or other light channel configured to transmit light from the at least one light emitter to the at least one light detector.

According to some embodiments, the tip detection system does not comprise at least one light pipe or other light channel configured to transmit light from the at least one light emitter to the at least one light detector. In some embodiments, a direct line of sight does not exist between the at least one light emitter and the at least one light detector. In some arrangements, the at least one light emitter is adjacent to the at least one light detector. In some embodiments, a direct line of sight exists between the at least one light emitter and the at least one light detector.

According to some embodiments, a method of controlling a skin treatment procedure comprises detecting a property of a tip secured to a handpiece of a skin treatment device according to any one of embodiments disclosed herein or equivalents thereof, and determining whether the skin treatment procedure should proceed or commence.

According to some embodiments, the method further comprises terminating the skin treatment procedure. In some embodiments, the method further comprises providing an alert (e.g., an audible alert, a visual alert, a haptic alert, etc.). In some embodiments, the method further comprises generating and/or sending an electronic communication.

According to some embodiments, a tip for use in a skin treatment device comprises a proximal portion configured to secure to a distal end of a handpiece of the skin treatment device, wherein the tip is configured to be placed in fluid communication with a fluid source when the tip is secured to the handpiece, a distal portion configured to contact a target skin surface during use, a fluid pathway placing the fluid source in fluid communication with the distal portion when the tip is secured to the handpiece, and at least one detectable portion configured to be within a light path of a tip detection assembly of the handpiece when the tip is secured to the handpiece, wherein the handpiece is configured to detect a property of at least one detectable portion of the tip when the tip is secured to the handpiece.

According to some embodiments, the at least one detectable portion of the tip is positioned along an interior of a stem of the tip. In some embodiments, the tip is disposable.

According to some embodiments, a handpiece for use in skin treatment procedure comprises a main body, a tip positioned along a distal end of the main body, at least one fluid conduit configured to fluidly connect the tip to a fluid source, at least one valve along the at least one fluid conduit, the at least one valve configured to regulate a flow of treatment fluid passing through the at least one fluid conduit, and at least one controller configured to adjust a position of the at least one valve to regulate a flowrate of treatment fluid transferred to the tip, wherein the at least one controller comprises at least one electronic component to facilitate a communication between the at least one controller and the at least one valve.

According to some embodiments, the at least one valve comprises an electronically controlled valve (e.g., valve controlled by a stepper motor, other type of motor, solenoid, other type of actuator, etc.). In one embodiment, the valve comprises a proportional solenoid valve. In some embodiments, the at least one valve comprises a pinch valve, a needle valve, a piezoelectric valve or any other type of valve or other flow control device or component.

According to some embodiments, the at least one controller comprises one or more buttons, switches, dials, knobs or any other component positioned along an exterior of the handpiece. In some embodiments, the at least one controller is not located on or in the handpiece. In some arrangements, the at least one controller is positioned on a separate system or computing device or component.

According to some embodiments, the handpiece further comprises at least one flowmeter configured to measure a flowrate of treatment fluid passing through the at least one fluid conduit. In some embodiments, the at least one flowmeter comprises a mechanical flowmeter. In some embodiments, the at least one flowmeter comprises an ultrasonic flowmeter.

According to some embodiments, the handpiece further comprises a force detection assembly configured to detect a force or a pressure exerted by the tip on a subject's skin surface during use. In some embodiments, the force detection assembly comprises at least one transducer or other pressure sensor.

According to some embodiments, the handpiece further comprises at least one output that is configured to provide an indication of the force or pressure exerted by the tip on a subject's skin surface. In some embodiments, the at least one output is positioned on the handpiece. In some embodiments, the at least one output is not positioned on the handpiece (e.g., the at least one output is located on a separate device or system). In some embodiments, the at least one output comprises a measured or estimated force or pressure. In some embodiments, the at least one output comprises a relative representation of a measured or estimated force or pressure. In some embodiments, the relative representation comprises an indication of “Low,” “Medium,” and “High.”

According to some embodiments, wherein the tip is removably secured to the main body.

According to some embodiments, the handpiece further comprises a tip detection assembly configured to automatically detect a type of tip that is secured to the handpiece. In some embodiments, the tip detection assembly is configured to detect differences in color or other property associated with light.

According to some embodiments, the handpiece further comprises a container identification detection assembly configured to automatically detect a vial or other container positioned within or near the handpiece. In some embodiments, the container identification detection assembly comprises a RFID reader that is configured to read information from a RFID tag positioned on a vial or other container. In some embodiments, the container identification detection assembly is configured to automatically detect a vial or other container, regardless of an orientation of the vial or container within or near the handpiece.

According to some embodiments, the handpiece further comprises a light module, the light module positioned at or near a distal portion of the handpiece and configured to deliver light toward a subject's skin surface. In some embodiments, at least one property of light being emitted from the light module can be modified. In some embodiments, the at least one property comprises color, wavelength, intensity, brightness, duration and/or the like.

According to some embodiments, the light module further comprises at least one camera or at least one sensor. In some embodiments, the at least one camera or the at least one sensor is configured to receive data regarding a subject's skin surface.

According to some embodiments, the handpiece further comprises a temperature detection module or assembly, wherein the temperature detection module or assembly is positioned along a distal end of the handpiece and is configured to detect a temperature.

According to some embodiments, the temperature detection module or assembly is configured to detect a temperature of a skin surface being treated. In some arrangements, the temperature detection module or assembly is configured to detect a temperature of the treatment fluid.

According to some embodiments, a handpiece for use in skin treatment procedure comprises a main body, a tip positioned along a distal end of the main body, at least one fluid conduit configured to fluidly connect the tip to a fluid source, at least one valve along the at least one fluid conduit, the at least one valve configured to regulate a flow of treatment fluid passing through the at least one fluid conduit, and at least one flowmeter configured to measure a flowrate of treatment fluid passing through the at least one fluid conduit.

According to some embodiments, the at least one flowmeter comprises a mechanical flowmeter. In some embodiments, the at least one flowmeter comprises an ultrasonic flowmeter. In some embodiments, the at least one flowmeter is located upstream of the at least one valve. In some embodiments, the at least one flowmeter is located downstream of the at least one valve.

According to some embodiments, a handpiece for use in skin treatment procedure comprises a main body, a tip positioned along a distal end of the main body, and a force detection assembly, said force detection assembly being configured to detect a force or a pressure exerted by the tip on a subject's skin surface during use. In some embodiments, the force detection assembly comprises at least one transducer or other pressure sensor.

According to some embodiments, the handpiece comprises at least one output that is configured to provide an indication of the force or pressure exerted by the tip on a subject's skin surface. In some embodiments, the at least one output is positioned on the handpiece. In some embodiments, the at least one output is not positioned on the handpiece. In some arrangements, the at least one output is located on a separate device or system. In some embodiments, the at least one output comprises a measured or estimated force or pressure. In some embodiments, the at least one output comprises a relative representation of a measured or estimated force or pressure. In one embodiment, the relative representation comprises an indication of “Low,” “Medium,” and “High.”

According to some embodiments, a handpiece for use in skin treatment procedure comprises a main body, a tip positioned along a distal end of the main body, and a container identification detection assembly configured to automatically detect a vial or other container positioned within or near the handpiece.

According to some embodiments, the container identification detection assembly comprises a RFID reader that is configured to read information from a RFID tag positioned on a vial or other container. In some embodiments, the container identification detection assembly is configured to automatically detect a vial or other container, regardless of an orientation of the vial or container within or near the handpiece.

According to some embodiments, a handpiece for use in skin treatment procedure comprises a main body, a tip positioned along a distal end of the main body, and a tip detection assembly configured to automatically detect a type of tip that is secured to the handpiece. In some embodiments, the tip detection assembly is configured to detect differences in color or other property associated with light.

According to some embodiments, a handpiece for use in skin treatment procedure comprises a main body, a tip positioned along a distal end of the main body, and a light module, the light module positioned at or near a distal portion of the handpiece and configured to deliver light toward a subject's skin surface.

According to some embodiments, at least one property of light being emitted from the light module can be modified. In some embodiments, the at least one property comprises color, wavelength, intensity, brightness, duration and/or the like. In some arrangements, the light module further comprises at least one camera or at least one sensor. In some embodiments, the at least one camera or the at least one sensor is configured to receive data regarding a subject's skin surface.

According to some embodiments, a handpiece for use in skin treatment procedure comprises a main body, a tip positioned along a distal end of the main body, and a temperature detection module or assembly, wherein the temperature detection module or assembly is positioned along a distal end of the handpiece and is configured to detect a temperature.

According to some embodiments, the temperature detection module or assembly is configured to detect a temperature of a skin surface being treated. In some arrangements, the temperature detection module or assembly is configured to detect a temperature of the treatment fluid.

According to some embodiments, a method for treating skin comprises delivering a treatment fluid to a tip of a handpiece, the handpiece comprising a main body and the tip, wherein the tip is positioned at or near a distal end of the main body, wherein the handpiece comprises at least one fluid conduit configured to fluidly connect the tip to a fluid source, and controlling at least one operational aspect of the handpiece using at least one controller of the handpiece, wherein the at least one controller comprises at least one electronic component to facilitate a communication between the at least one controller and at least one component of the handpiece.

According to some embodiments, a method for treating skin comprises wherein the at least one operational aspect comprises a flowrate of the treatment fluid to the tip. In some embodiments, the at least one controller comprises one or more buttons, switches, dials, knobs or any other component positioned along an exterior of the handpiece. In some arrangements, the at least one controller is not located on or in the handpiece. In other embodiments, the at least one controller is positioned on a separate system or computing device or component.

According to some embodiments, a method for treating skin comprises delivering a treatment fluid to a tip of a handpiece, the handpiece comprising a main body and the tip, wherein the tip is positioned at or near a distal end of the main body, wherein the handpiece comprises at least one fluid conduit configured to fluidly connect the tip to a fluid source, and measuring or approximating a flowrate of treatment fluid passing through the at least one fluid conduit using a flowmeter. In some embodiments, the method further comprises regulating a flow of treatment fluid passing through the at least one fluid conduit. In some embodiments, the handpiece comprises at least one valve along the at least one fluid conduit, the at least one valve configured to modify a flow of treatment fluid passing through the at least one fluid conduit. In some embodiments, the at least one flowmeter comprises a mechanical flowmeter. In some embodiments, the at least one flowmeter comprises an ultrasonic flowmeter.

According to some embodiments, a method for treating skin comprises performing a skin treatment procedure using a tip of a handpiece, the handpiece comprising a main body and the tip, wherein the tip is positioned at or near a distal end of the main body, and measuring or approximating a force or a pressure exerted by the tip on a subject's skin surface during use, wherein said measuring or approximating a force or a pressure is performed using a force detection assembly of the handpiece.

According to some embodiments, the force detection assembly is positioned at least partially on or in the handpiece. In some embodiments, the force detection assembly comprises at least one transducer or other pressure sensor.

According to some embodiments, the method for treating skin further comprises providing an indication of the force or pressure exerted by the tip on a subject's skin surface using at least one output. In some embodiments, the at least one output is provided on the handpiece. In other arrangements, the at least one output is not provided on the handpiece. In some embodiments, the at least one output is located on a separate device or system. In one embodiment, the at least one output comprises a measured or estimated force or pressure. In some arrangements, the at least one output comprises a relative representation of a measured or estimated force or pressure. In one embodiment, the relative representation comprises an indication of “Low,” “Medium,” and “High.”

According to some embodiments, a method for treating skin comprises performing a skin treatment procedure using a tip of a handpiece, the handpiece comprising a main body and the tip, wherein the tip is positioned at or near a distal end of the main body, and automatically detecting a vial or other container positioned within or near the handpiece using a container identification detection assembly of the handpiece.

According to some embodiments, the container identification detection assembly comprises a RFID reader that is configured to read information from a RFID tag positioned on a vial or other container. In some embodiments, the container identification detection assembly is configured to automatically detect a vial or other container, regardless of an orientation of the vial or container within or near the handpiece.

According to some embodiments, a method for treating skin comprises performing a skin treatment procedure using a tip of a handpiece, the handpiece comprising a main body and the tip, wherein the tip is positioned at or near a distal end of the main body, and automatically detecting a type of tip that is secured to the handpiece using a detection assembly of the handpiece. In some embodiments, the tip detection assembly is configured to detect differences in color or other property associated with light.

According to some embodiments, a method for treating skin comprises performing a skin treatment procedure using a tip of a handpiece, the handpiece comprising a main body and the tip, wherein the tip is positioned at or near a distal end of the main body, and selectively delivering light toward a subject's skin surface using a light module, the light module positioned at or near a distal portion of the handpiece.

According to some embodiments, the method further comprises modifying at least one property of light being emitted from the light module. In some embodiments, the at least one property comprises color, wavelength, intensity, brightness, duration and/or the like. In some embodiments, the light module further comprises at least one camera or at least one sensor. In some embodiments, the at least one camera or the at least one sensor is configured to receive data regarding a subject's skin surface.

According to some embodiments, a method for treating skin comprises performing a skin treatment procedure using a tip of a handpiece, the handpiece comprising a main body and the tip, wherein the tip is positioned at or near a distal end of the main body, and measuring or approximating a temperature at, along or near a distal end of the handpiece using a temperature detection module or assembly, wherein at least one component of the temperature detection module or assembly is located at, along or near the tip.

According to some embodiments, a method for treating skin comprises performing a skin treatment procedure using a tip of a handpiece, the handpiece comprising a main body and the tip, wherein the tip is positioned at or near a distal end of the main body, delivering a treatment fluid to the tip using at least one fluid delivery conduit, wherein at least one valve in fluid communication with the at least one fluid delivery conduit is configured to regulate a flow of treatment fluid passing through the at least one valve, generating a vacuum or positive pressure using a vacuum or positive pressure source fluidly coupled to the tip, and controlling a position of the at least one valve, and at least one operational aspect of the vacuum or positive pressure (e.g., a vacuum or positive pressure amount) using at least one controller, wherein the at least one controller comprises an electronic controller.

According to some embodiments, the at least one controller comprises a single controller. In other embodiments, the at least one controller comprises a first controller for controlling the position of the at least one valve, and a second controller for controlling at least one operational aspect of the vacuum or positive pressure.

According to some embodiments, the handpiece comprises or is operatively coupled to a processor, the processor configured to help control the position of the at least one valve and/or the at least one operational aspect of the vacuum or positive pressure.

According to some embodiments, the processor is configured to help control the position of the at least one valve and/or the at least one operational aspect of the vacuum or positive pressure based on a closed-loop or open-loop configuration or scheme.

According to some embodiments, a handpiece for use in skin treatment procedure comprises a main body, a tip positioned along a distal end of the main body, at least one fluid conduit configured to fluidly connect the tip to a fluid source, and at least one controller configured to adjust at least one parameter associated with an operation of the handpiece, wherein the at least one controller comprises at least one electronic component.

According to some embodiments, the at least one controller comprises one or more buttons, switches, dials, knobs or any other component positioned along an exterior of the handpiece. In some embodiments, the at least one controller is controlled by manipulation, by audible command and/or by gesture control.

According to some embodiments, the at least one parameter associated with an operation of the handpiece comprises one or more of the following: selection of a flowrate of fluid passing through the at least one fluid conduit, selection of the fluid source to be delivered to the handpiece, selection of a vacuum force delivered to the tip during use, and/or regulation of an aspect of at least one separate modality.

According to some embodiments, the at least one separate modality comprises at least one of a light delivery element, an imaging element, a temperature modification element and/or the like.

### BRIEF DESCRIPTION OF THE DRAWINGS

These and other features, aspects and advantages of the present application are described with reference to drawings of certain embodiments, which are intended to illustrate, but not to limit, the present inventions. It is to be understood that these drawings are for the purpose of illustrating the various concepts disclosed herein and may not be to scale.

FIG. 1 illustrates a side view of a handpiece for skin treatment according to one embodiment;

FIG. 2A schematically illustrates a flow diagram for a skin treatment system according to one embodiment;

FIG. 2B schematically illustrates a flow diagram for a skin treatment system according to another embodiment;

FIG. 3 illustrates a side view of a handpiece for skin treatment according to another embodiment;

FIG. 4A schematically illustrates a cross-sectional view of a handpiece configured for use in a skin treatment system, according to one embodiment;

FIG. 4B schematically illustrates a cross-sectional view of a handpiece configured for use in a skin treatment system, according to another embodiment;

FIG. 5 illustrates a perspective view of a handpiece comprising a force detection assembly according to one embodiment;

FIGS. 6A and 6B illustrate embodiments of a force/pressure sensing output for a handpiece or other skin treatment device;

FIG. 7A illustrates a distal end of a handpiece comprising force detection assembly according to one embodiment;

FIG. 7B illustrates a cross-sectional view of the handpiece of FIG. 7A;

FIG. 8A illustrates a front view of a tip detection assembly for a handpiece according to one embodiment;

FIG. 8B illustrates a side/cross-sectional view of the assembly of FIG. 8A;

FIG. 8C illustrates a partial cross-sectional view of a tip detection assembly for a handpiece according to another embodiment;

FIGS. 9A to 9C illustrate various partial cross-sectional views of a handpiece comprising an identification tag reader according to one embodiment;

FIG. 10 illustrates different views of a handpiece having enhanced light delivery/detection capabilities according to one embodiment;

FIG. 11 illustrates a front perspective view of a handpiece having enhanced light delivery/detection capabilities according to another embodiment; and

FIG. 12 illustrates a side perspective view of a temperature detection module or assembly for use on a handpiece according to one embodiment.

### DETAILED DESCRIPTION

Although the various embodiments of a handpiece assembly (e.g., handpiece, handheld device, etc.) have specific relevance to a skin treatment system, the features, advantages and other characteristics disclosed herein may have direct or indirect applicability in other applications, such as, for example, medical devices (e.g., for skin treatment purposes, for other purposes, etc.), mechanical devices (e.g., medical or non-medical devices, industrial devices, retail products, etc.) and/or the like.

Several embodiments of the inventions disclosed herein are particularly advantageous because they include one, several or all of the following benefits: provide a handpiece for use in a skin treatment system that provides one or more features that enhance the execution of a corresponding treatment procedure, facilitate a treatment procedure for the aesthetician or other user, provide a more comfortable and pleasant experience for the subject that is undergoing the skin treatment procedure and/or the like; provide for enhanced delivery of treatment fluids to the skin of a subject; provide for more accurate delivery of fluids to the skin surface being treated; facilitate the delivery of fluids to the skin of a subject; provide the ability to accurately detect a flowrate of fluids being delivered to the skin surface; permit a measurement of force applied by a handpiece against the skin surface; enhance the efficacy, safety and comfort by using data collected regarding an amount of force being applied against the skin surface during a procedure; automatically detect the type of tip that is secured to a handpiece and ensure that it is appropriate to be used in a particular protocol; automatically detect a vial or other container inserted within a handpiece or other skin treatment device; detect and/or monitor a temperature at, along or near the tip and/or the skin surface; activate blue light, other types of light and/or the like using the handpiece or other device; detect images and/or sense light features using the handpiece; provide for enhanced collection of data regarding a skin treatment procedure; provide for enhanced treatment protocols based on data collection and processing; and provide for enhanced safety and other counterfeiting measures related to fluids delivered by skin treatment systems.

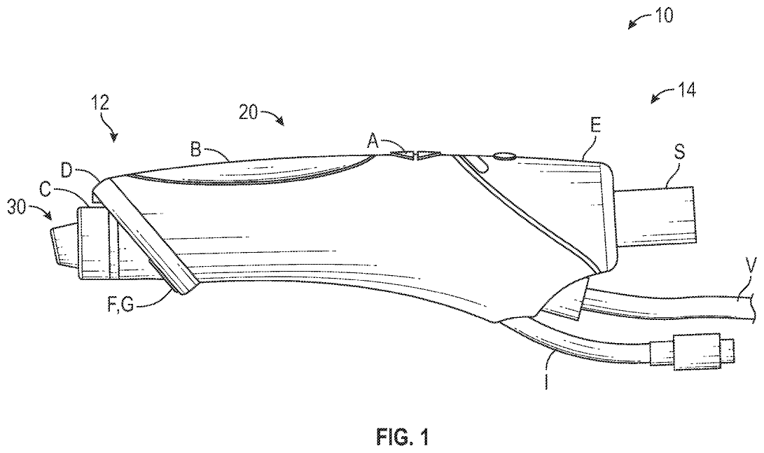

FIG. 1 illustrates one embodiment of a handpiece or other device 10 configured to be used for skin treatment. As shown, the handpiece or device 10 can include a main body or main body portion 20 and a tip 30 secured along the distal end 12 of the main body 20. In some embodiments, the tip 30 comprises a separate component or device that is configured to be removably secured to the main body 20. However, in other arrangements, the tip 30 and the main body 20 can form a unitary, monolithic and/or non-separable body or other configuration, as desired or required.

As noted herein and with continued reference to FIG. 1, the handpiece 10 can include one or more components and/or features that provide certain advantages and benefits. At least some of these components and features are schematically illustrated in FIG. 1 using various general callouts. By way of example, the handpiece can comprise electronic control of treatment materials and other fluids that are being transferred to the tip 30 (e.g., control of one or more aspects related to the delivery of such fluids and/or other materials, etc.), and thus, the skin surface being treated, using the handpiece 10. This is represented by callout A in the schematic illustrated in FIG. 1.

With continued reference to FIG. 1, in some embodiments, as represented by callout B, the handpiece or device 10 is configured to measure (e.g., precisely, within a particular tolerance (e.g., 0 to 5%, 0 to 10%, 0 to 20%, 5 to 20%, values between the foregoing, etc.) approximately, etc.) the flowrate of treatment materials or other fluids being transferred to the tip 30. This can assist with ensuring that the proper amount of treatment fluid is being delivered to the tip, and thus, the skin surface being treated, during a procedure. Relatedly, this can also reduce or eliminate the unnecessary and/or inadvertent waste (e.g., overuse) of certain treatment fluids and/or other materials.

Further, in some arrangements, the device 10 is configured to detect (e.g., measure, approximate, etc.) a force or pressure being applied by the user of the device to a subject's skin surface. This is represented by callout C in the schematic embodiment illustrated in FIG. 1. Force measurement can help ensure that a particular procedure is being performed using an appropriate amount of force applied by the handpiece or device 10 to or against the targeted skin surface. This can help improve a skin treatment procedure and/or help avoid injury to the subject undergoing the procedure.

With continued reference to FIG. 1, the handpiece or device 10 can be configured to detect (e.g., automatically, at least partially automatically, etc.) the type of tip 30 that has been secured along the distal end 12 of the handpiece 10. Such detection can occur once the tip has been secured to the handpiece. In some embodiments, as discussed in greater detail below, the device 10 is adapted to detect differences in color to distinguish between tips. However, in other arrangements, one or more other characteristics of the tips 30 can be used to allow the device 10 to identify (e.g., automatically) the tip, either in lieu of or in addition to color. For example, one or more of the following properties of the tips can be used for purposes of detection: translucency, refractive properties, weight, RFID or other identifiers included on or within the tips and/or the like, as desired or required.

In some arrangements, the handpiece or device 10 is configured to automatically detect and identify an identifier (e.g., a RFID tag) positioned on a vial or other fluid source component S secured to the handpiece 10. In some embodiments, such an identifier (e.g., a RFID tag, other tag, etc.) is detected by the handpiece or device 10 when the vial or other fluid source component S is at least partially positioned within (e.g., fully inserted or otherwise positioned, partially inserted or otherwise positioned, etc.) the corresponding slot, recess and/or other opening of the handpiece. This is generally represented by callout E in FIG. 1. However, in other embodiments, the tag or other identifier is detected when the vial is placed along, near, adjacent and/or otherwise in proximity (e.g., contact, non-contact proximity (e.g., within a particular distance, etc.)) of the handpiece or other component of the corresponding skin treatment system (and/or a device or system (e.g., tablet, smartphone, etc.) that is operatively coupled to the handpiece and/or other components of the skin treatment system. Such embodiments can be configured to identify one or more RFID tags or other identifiers (e.g., other electromagnetic identifiers, graphic or other visual identifiers such as QR codes, barcodes, alphanumeric codes, etc.) secured to or otherwise associated with a serum or other treatment fluid container. The tags or other identifiers can help ensure that the correct serums and other treatment serums are being utilized for a specific skin procedure or other protocol.

For any of the embodiments disclosed herein, the handpiece and/or the skin treatment system (e.g., a system that includes the handpiece) can include a processor (e.g., central processing unit) that receives data from the detector(s) E. In some arrangements, the processor is able to receive data from detector(s), process it, and execute a set of commands (e.g., algorithm, etc.) which can at least in part be based on the data received from the detector(s) E for the fluid source component S, or other detector(s) within, associated with, or operatively connected to the handpiece and/or the skin treatment system. The processor can include storage to store data collected from the detector(s) E and similarly process it, and execute a set of commands (e.g., algorithm, etc.) which can at least in part be based on the data received from the detector(s).

As a non-limiting example, the detector(s) E can read data stored within or pulled from a table called out by the identifier positioned on the vial or other fluid source component S secured to the handpiece 10, and perform one or more of the following actions: provide information regarding the contents of the container, such as the expiration date, manufacturing date, size, lot number, skin procedure with which the contents are intended to be used, and other information related to the specific fluid within the vial or other fluid source component S; pick, based on the type of fluid recorded to be present within the fluid source component S, one or more operational patterns for a procedure; collect data regarding the use of and/or related to the corresponding fluid source S can be gathered or otherwise collected to generate reports for billing, reordering, recording use of that specific vial (if applicable), and/or other purposes; recording the number of times that a vial or other container has been removed and reinserted within a handpiece 10, and/or limiting or otherwise affecting at least one operational parameter of the handpiece 10 if the recorded number exceeds a certain number; determine if a rinse, flush and/or other steps or actions are required before the fluid from that vial or other fluid source component S can be used and/or any other action can be taken in connection with use of the device; or other operations as disclosed herein.

Another feature of the handpiece 10, as represented by callout F and G in FIG. 1, includes integrated imaging and integrated light delivery. The incorporation of a camera or other light sensing device or component into the handpiece 10 and the ability to deliver light can improve detection, skin health evaluation and assessment, chemical activation and/or the like.

As embodied by general callout I in the illustrated configuration, the device 10 can include the necessary electrical, electronic, mechanical, electromechanical and/or other components or features to assist with the implementation of one or more of the foregoing features into its design. Such components or features can include, without limitation, wiring, batteries or other power-related components, chips, buttons, switches or other controllers, control units or processors, memory components, valves, sensors and/or the like, as desired or required.

With continued reference to FIG. 1, for any of the embodiments disclosed herein or equivalents thereof, a handpiece or device 10 can be configured to transfer treatment materials and other fluids from one or more containers or other fluid sources S (e.g., a vial, a cartridge that is in fluid communication with a larger manifold system (not shown), etc.) through one or more conduits (also not shown) passing, at least partially, through an interior of the main body 20 of the device 10. The device 10 can be configured to be placed in fluid communication with a vacuum or suction source (e.g., via one or more vacuum conduits V). The handpiece 10 can be configured such that the application of vacuum or suction creates, at least in part, a vacuum or suction force along the tip 30. In some embodiments, a tip and/or other distal portion of the handpiece includes a peripheral member that is configured to contact skin tissue during use and define or circumscribe an interior area. In some embodiments, when contact is made between the tip or other distal portion of a handpiece and targeted skin tissue, a temporary seal is created between said tip or the handpiece, allowing a vacuum or suction force created therein to help “draw,” “pull” or otherwise deliver or transfer fluids from a fluid source (e.g., a vial, a multi-container manifold or similar component/system) to such an interior area (and thus, the interface between the skin and the device (e.g., tip, handpiece, etc.)).

The application of suction or vacuum at, near or along the tip 30 or other portion of the distal end 12 of the device 10 can assist with removing spent fluid and/or debris (e.g., sebum, dirt, oils, exfoliated skin or other tissue of the subject being treated, etc.) away from the working end of the device 10 during use. Further, in some arrangements, the application of vacuum or suction along the working end can help draw treatment materials and/or other fluids (e.g., acidic solutions, serums, etc.) to the working end. For instance, treatment fluids can be transferred from a fluid source S (e.g., a vial, a manifold system, etc.) to the tip 30 when a seal is formed (or substantially formed) between the tip 30 and a portion of the skin surface being treated. Under such circumstances, the application of vacuum or suction can help draw treatment fluids through one or more ports of the tip and via one or more fluid conduits placing the fluid source S in fluid communication with the tip 30.

For any of the embodiments disclosed herein, the tip 30 or other portion of the distal end 12 of the device 10 that is configured to contact skin tissue can be configured to at least partially exfoliate and/or otherwise remove skin tissue when the device is in use. In some embodiments, the tip 30 and/or other portion of or near the distal end 12 of the device 10 comprises features (e.g., edges, protruding components, ridges, etc.) that assist with skin removal. For example, such features can help remove skin when the tip 30 or other portion of the distal end 12 of the device 10 is moved (e.g., translated) relative to the targeted skin surface. In some arrangements, the efficacy and/or extent of exfoliation or other skin removal is enhanced by the application of suction or vacuum along the working end (e.g., tip, distal end, etc.) of the device.

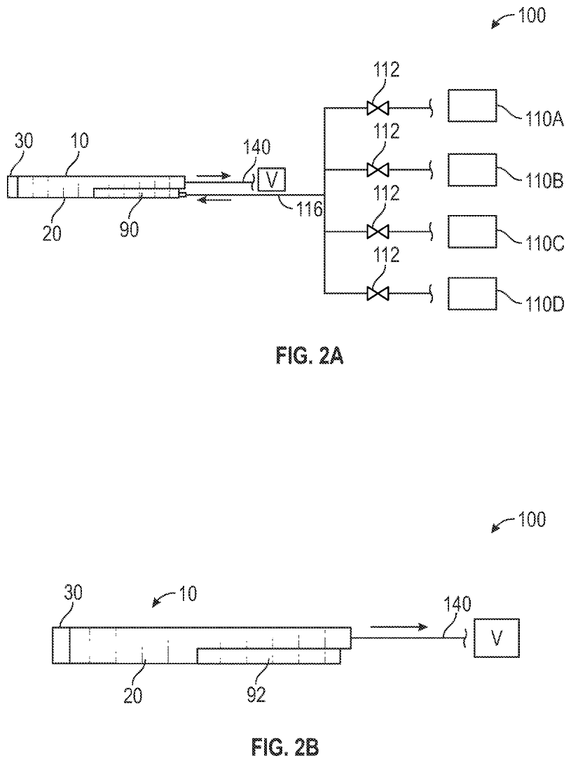

According to certain embodiments, as schematically illustrated in FIG. 2A, one or more bottles or other containers 110A, 110B, 110C, 110D can be placed in fluid communication with a manifold assembly 100. In some arrangements, as illustrated in FIG. 2A, one or more valves or other flow control devices or components 112 can be positioned between each fluid source (e.g., a bottle) and a main delivery conduit or other fluid line 116 that places the manifold assembly 100 in fluid communication with a handpiece 10. The valves or other flow control devices 112, which can include 2-way valves, can be configured to regulate the flow of treatment materials from one or more of the bottles or other containers 110A, 110B, 110C, 110D to the main delivery conduit 116, and thus, to the device 10.

With continued reference to FIG. 2A, the handpiece 10 can be placed in fluid communication with a suction source (e.g., vacuum pump, not shown) using one or more vacuum or suction conduit or other fluid line 140. In the arrangements disclosed herein, the manifold assembly 100 comprises a total of four fluid branches. However, a system can comprise more or fewer fluid branches (e.g., 1, 2, 3, 4, 5, 6, 7, 8, more than 8, etc.), as desired or required by a particular application or use. Further, one or more of the bottles or other containers 110A, 110B, 110C, 110D that are positioned within the manifold assembly 100 can include a cleaning solution (e.g., for the periodic flushing and cleaning of the manifold assembly).

According to certain embodiments, one or more of the fluid conduits of the manifold system illustrated in FIG. 2A are configured to provide a serum, other treatment fluid and/or the like. Alternatively, however, one or more of the conduits can be configured to receive water (e.g., distilled, tap water, filtered, etc.), saline, other dilutants or dissolvents, other fluids and/or the like to the handpiece assembly 50. Such fluids can be adapted to contact and dissolve, dilute, liquefy, soften and/or otherwise mix with one or more solids, gels and/or other materials positioned within or on various surfaces or portions of the handpiece 10 (e.g., tip 30 or distal end 12). This can provide a convenient method of providing one or more materials to the skin-tip interface and/or any other location where such materials are desired or required.

Further, as illustrated schematically in FIG. 2B and discussed in greater detail herein, for any device or system disclosed herein or equivalent thereof, the device or system can be configured to permit a user (e.g., an aesthetician, a cosmetician, a dermatologist, etc.) to (1) connect a handpiece 10 to a manifold assembly 100 (e.g., using a “dummy” cartridge 90, as shown in FIG. 2A), and/or to (2) insert an actual fluid or treatment material-containing cartridge 92 directly into the handpiece assembly, e.g., a recess of the handpiece assembly (as shown in FIG. 2B). These two options can be used interchangeably to perform a particular treatment procedure or protocol, as desired or required.

### Control Features

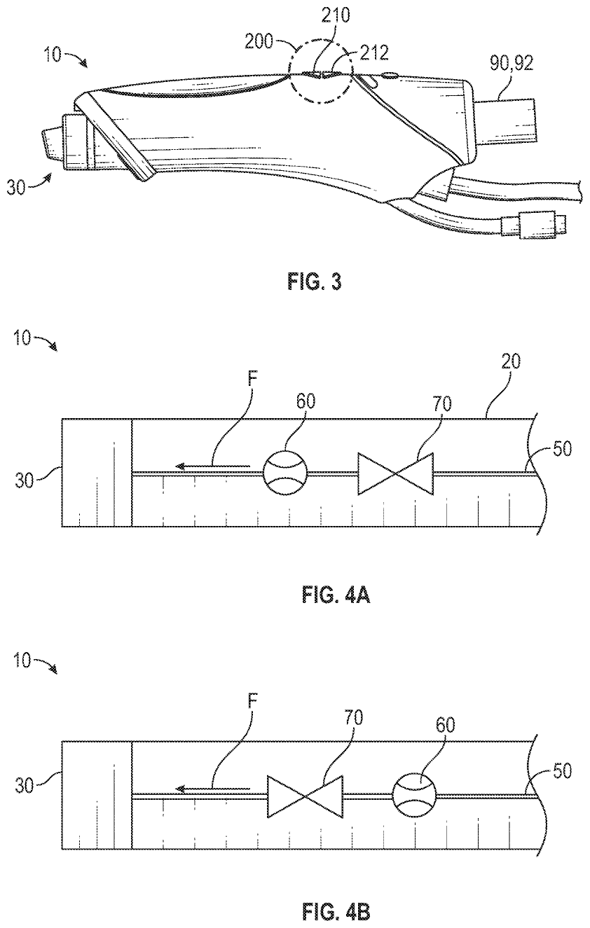

FIG. 3 illustrates one embodiment of a handpiece 10 that includes one or more buttons or other controllers 210, 212 that are, at least in part, electronically controlled or otherwise operated. Such buttons or other controllers 210, 212 can be located along any area or portion 200 of the handpiece or device 10. For example, in some arrangements, the buttons or controllers 210, 212 are positioned along the top of the main portion 20. The buttons 210, 212 can be located and otherwise configured to advantageously permit the user to manipulate them with single hand control (e.g., without the need for the user to reposition or otherwise move his or her hand during use).

The buttons or other controllers 210, 212, and/or the area 200 in or around which the buttons or controllers are positioned can be configured to have the necessary resistance to liquids (e.g., water, serums, other treatment materials, etc.), other substances, ambient conditions and/or occurrences and/or the like. For example, in some embodiments, the system is manufactured to be waterproof or water resistant. The system can be designed and otherwise manufactured to satisfy certain waterproofing standards. By way of example, the system can be configured to meet the requirements of IPxx, the ingress protection (IP) protection grade system drafted by IEC (International Electrotechnical Commission), which classifies electrical appliances according to their dust and moisture resistance. In some embodiments, the system complies with one or more IPxx standards (e.g., IP22).

As noted above, the device 10 can be designed, manufactured and otherwise configured to meet certain standards with respect to drops, impacts, vibrations and the like. Further, in some embodiments, the system and/or its components (e.g., housing, internal components, etc.) are configured to include a temperature resistance of not only ambient conditions (e.g., 10 to 40 degrees C), but also relatively extreme temperature conditions (e.g., −10 degrees C to 50 degrees C). Further, the handpiece or other device 10 can be configured to provide the necessary resistance to oils, chemicals and/or other materials to which a user may expose the system. For instance, the handpiece 10 can be adapted to protect the housing and/or other system components against liquids and/or other materials used during the frequent or infrequent cleaning or sanitization of the system. According to some embodiments, the system can be configured to sustain a drop from 1 meter or more (e.g., 1 m, 1.5 m, 2 m, 0 m to 1 m, 0 m to 2 m, 1.5 to 2 m, values between the foregoing ranges or values, etc.).

In some embodiments, the device 10 comprises a touchscreen or other similar component, and not necessarily distinct buttons, switches or other controllers. The device 10 can include one, two, three or more buttons or controllers 210, 212, as desired or required by a particular application or use. For example, in the arrangement illustrated in FIG. 3, the controller area 200 of the device 10 can include buttons 210, 212 that regulate the flow of treatment materials or other fluids being transferred from a fluid source (e.g., a fluid vial 92, a cartridge 90 configured to be placed in fluid communication with a manifold system 100 as schematically illustrated in FIG. 2A, etc.) secured to the handpiece. In some embodiments, the buttons 210, 212 control the selection of treatment fluid from the fluid source (e.g., a bottle secured to a manifold or other fluid distribution/control system, a vial secured to the handpiece, etc.). For example, an operator could toggle from drawing fluid from fluid source 110A to drawing fluid from fluid source 110B by pressing button 210 or button 212. In some embodiments, toggling buttons 210, 212 could also change one or more operational parameters of the handpiece 10 instead of, or in addition to changing the fluid source. Is some arrangements, two or more buttons or controllers can be selected (e.g., manually, audibly, etc.) to activate the delivery of fluids from two or more bottles secured to the system to the handpiece (e.g., sequentially, simultaneously, etc.). In some embodiments, the ability to change and/or otherwise select a fluid source or alter operational parameters of the system (e.g., to increase or decrease level of vacuum, activate/deactivate pulsation, adjust the flowrate of fluids being delivered to the handpiece and tip, activation/deactivation of light, other modalities and functionalities disclosed herein and/or the like) can provide one or more benefits (e.g., increase the efficiency and/or efficacy of the newly selected fluid source, permit for specifically tailored or customized treatment approaches based on, for example, the physical parameters and associated treatment use of the newly selected fluid source, make the use of treatment fluids more efficient, reduce waste, facilitate the execution of a skin treatment procedure and make the process more comfortable and enjoyable for both the user and the subject, improve safety, etc.). As illustrated in FIG. 3 and other figures provided in the present application, such cartridges or vials 90, 92 can be sized, shaped and otherwise adapted for removable placement into a recess or other opening of the device 10. In some embodiments, one button 210, when actuated (e.g., pressed), is configured to increase the flowrate of treatment materials to the tip 30, whereas another button 212, when actuated, is configured to decrease such a flowrate. In other arrangements, however, a device 10 can include more or fewer buttons or other controllers. Such controllers 210, 212 can be configured to help control any other aspect of the operation of the device and/or any other devices, components, systems, etc. that may be operatively coupled and/or otherwise used together with the device. Such controllers can be used to advantageously control or regulate pressure (e.g., pressure of fluids being delivered to the tip), vacuum or suction pressure applied to the device, and/or activation/deactivation of other components of the device (e.g., imaging, light emitting components, force sensing, etc.), as desired or required. For example, in some embodiments, a processor operatively coupled to such controllers can be configured to receive data (e.g., from force or pressure sensors, other sensors, etc.) and based on, for example, force or pressure measurements, temperature measurements, elapsed time, one or more characteristics of the subject being treated (e.g., age, gender, skin health, skin type, skin conditions, intended goals, etc.), data capture and analysis, tip type and/or the like, can alter or otherwise modify one or more operational parameters of the system and the related treatment process.

In some embodiments, one or more controllers are positioned on and/or within the handpiece. In some arrangements, one or more controllers are not positioned on and/or within the handpiece. For example, a controller can be positioned on a console or tower system, a separate computing device and/or the like, as desired or required.

In some embodiments, the controller of the handpiece can be operatively coupled to a processor (e.g., a microprocessor, a microcontroller (e.g., or microcontroller unit (MCU), etc.). Such a processor can include one or more chips, a processor (e.g., a CPU), a memory and/or the like. In some embodiments, the processor can be integrated with the controller. However, in other embodiments, the processor can be separate from the controller. For example, the processor can be included on and/or in the handpiece but not directly incorporated into or secured to one or more other components of the controller. In yet other embodiments, the processor is operatively coupled to a controller but is not located in the handpiece. For instance, the processor can be included with a tower or console, a separate computing device and/or the like, as desired or required. In such embodiments, the processor can be operatively coupled to a controller via one or more wired and/or wireless connections or technologies. In some arrangements, the controller and/or another portion or component of the handpiece comprises and/or is operatively coupled to (e.g., mechanically, electrically, etc.) a stepper motor, a driver, an actuator and/or any other component.

In some embodiments, the device can be configured to receive commands using other types of commands besides those provided by manipulating one or more buttons or other controllers. For instance, the handpiece can be configured to receive audible commands of the user, can include gesture control and/or the like.

As noted above, the buttons or other controllers 210, 212 of the device 10 can include one or more electric and/or electronic components. For example, the controllers can be configured to regulate the opening and/or closing (e.g., partially or completely) of one or more valves that are in the fluid pathway connecting the tip 30 or distal end 12 of the device to the fluid source S. Examples of such a valve 70 are schematically depicted in FIGS. 4A and 4B. As shown, a valve 70 can be located within an interior of the main body 20 of the handpiece 10. For any of the embodiments disclosed herein, a valve or other flow-regulating devices, component or feature of the device can include a pinch valve, a needle valve, a ball valve, a piezoelectric valve, and/or any other type of valve. The controller(s) 210, 212 (e.g., button(s), knob(s), dial(s), touchscreen(s), etc.) can be electrically coupled to such valves or other components to regulate the valves or components in accordance with the actuation of such controller(s) by the aesthetician or other user. In some embodiments, the valve, irrespective of type and other details, can include and/or be operatively coupled to one or more other components. For instance, one or more of the following can be included (e.g., integrated with the valve or valve assembly, included in the handpiece, etc.) to facilitate with the operation and function of the valve or other flow control device or member: an actuator, a motor, a solenoid, a processor, wired and/or wireless connections, a memory, a power source, other electronic components and/or the like. In some embodiments, the valve or valve assembly is electronically controlled. For example, the valve or valve assembly can include a stepper motor with a valve or a solenoid proportional valve. The controller(s) can be coupled to one or more valves and/or other components using hardwired and/or wireless connections, as desired or required.

According to some embodiments, the handpiece can include one or more controllers (e.g., electronic controllers, electronic controller assemblies, etc.) and/or similar components or devices that are configured to regulate one or more aspects of vacuum or suction force (e.g., negative pressure) and/or positive pressure that is delivered to the tip or other handpiece/skin tissue interface during use. As discussed herein, the handpiece can be placed in fluid communication with one or more vacuum or suction sources and/or positive pressure sources (e.g., pump, compressor, etc.). In some embodiments, the handpiece can include buttons and/or other controllers that can be manipulated to regulate the amount of pressure (e.g., positive and/or negative relative to atmospheric), manner of delivery (e.g., constant, intermittent, pulsing, etc.), frequency (e.g., for non-constant delivery) and/or other properties or characteristics of fluid (e.g., air, treatment liquid, other fluids, etc.) delivery to the distal end (e.g., tip) of the handpiece. For example, one or more controllers can vary the amount of negative and/or positive pressure of fluid delivery, whether such fluid is delivered in a constant, non-constant or varying (e.g., step-wise, sinusoidal, etc.) manner, the frequency of fluid delivery, start/stop/pause functions, etc.

In some embodiments, a handpiece includes both at least one controller to vary a position of a valve or other flow control device and at least one controller to vary a property of vacuum or positive pressure fluid delivery. However, in other embodiments, a handpiece can include an integrated controller that is configured to regulate at least one property of the fluid (e.g., air) suction/delivery (e.g., using vacuum and/or positive pressure), the position of a valve or other flow control device or feature and/or any other aspect of the fluidic system of the handpiece or a larger treatment system. Such control schemes can ensure that certain operational parameters of the handpiece are maintained within desired values and/or ranges. Such control schemes can help ensure that the system operates in a safe and/or efficacious manner. For instance, in some embodiments, a valve can be configured to open (e.g., allowing a greater volume of fluid to pass through it) when the vacuum pressure along the tip is relatively low. This can be done to maintain a minimum flowrate of treatment fluid being delivered to the tip/skin interface during use (e.g., to prevent an overly dry procedure). Likewise, the valve can close when the vacuum applied to the tip is increased (e.g., to prevent too much fluid being delivered to the tip/skin interface during use). In other embodiments, one or more properties of the vacuum or positive pressure being delivered to the tip can be modified according to the position of the valve. The handpiece can include one or more processors that help regulate aspects of the valve and/or the vacuum/positive pressure system according to an algorithm.

For any of the embodiments disclosed herein, the handpiece can be configured to operate in an open-loop or closed-loop configuration or scheme. For instance, the handpiece/skin treatment system can be configured to receive one or more user commands or other inputs (e.g., through a touchscreen, audible command, gesture control, etc.), can use sensor feedback (e.g., data obtained, manually or automatically, using one or more sensors or other devices (e.g., flowrate sensors, pressure sensors, imaging device, imaging sensors, position sensors, etc.) and/or other inputs) to at least partially automatically adjust one or more operational parameters of the handpiece and/or a larger skin treatment system. Such parameters can include, without limitation, control treatment fluid flow (e.g., by adjusting a valve position, by adjusting amount of vacuum/positive pressure of air or other fluid at or along the tip, etc.), vary one or more parameters of vacuum/pressure applied to the tip or other distal end of the handpiece, vary one or more aspects of temperature regulation elements included in the handpiece or overall system and/or the like. Such feedback schemes can be implemented to achieve a desired performance of the device/system during use, to reduce the likelihood of harm, damage or other undesirable results, etc.

Although controllers are disclosed herein in connection with only certain functions, such as, for example, fluid flow control, vacuum pressure and/or the like, controllers (e.g., buttons, other manipulatable components, features or devices, etc.) can be used to control and/or otherwise regulate (e.g., electronically) one or other parameters, functions, features, etc. of a handpiece (e.g., in connection with flow control, flow measurement, tip detection, vial/container identification, light emission, imaging or detection and/or the like, as desired or required.

### Flow Measurement

For any of the embodiments disclosed herein, a handpiece or device 10 can be configured to measure or approximate a flowrate of treatment material or other fluid being transferred to the tip 30 or distal end 12 using the device 10. As noted above, the measurement of flowrate can provide one or more benefits or advantages to the handpiece, and thus, a skin treatment procedure in which the handpiece is used. For instance, an accurate or even approximate measurement or estimation of flowrate can help ensure that a proper amount of treatment material or other fluid is being delivered to the skin surface during a procedure. This can improve the efficacy of a particular procedure. It can also help ensure that the amount of treatment material and/or fluid being transferred to the tip 30 or distal end 12 of the device 10 is appropriate (e.g., within a designated range, not too low or too high, etc.) to ensure the safety of the subject undergoing the treatment procedure and/or the aesthetician or other user of the device. For example, the device 10 can be configured to abort a procedure, provide a warning to the user and/or take one or more other actions if the flowrate (or amount of fluid being delivered to the skin surface) is below a particular threshold level.

FIGS. 4A and 4B schematically illustrate two different embodiments of handpiece 10 comprising a flowmeter or other flow measurement or flow estimation device, system or component (e.g., sensor) 60. As shown in FIG. 4A, the flowmeter or other flow measurement device or component 60 can be located downstream of a valve or other flow regulating device or component 70 (and/or other hydraulic device or component). However, in other arrangements, as depicted in FIG. 4B, the flowmeter or other flow measurement device or component 60 can be located upstream of a valve or similar flow regulating device or component, as desired or required. In the illustrated embodiments, the direction of flow of the treatment material or other fluid being transferred to the tip 30 is generally depicted by arrow F.

Any flowmeter or other flow measurement device 60 can be included directly or indirectly in the fluid path of the device, such as, one or more fluid conduits 50 hydraulically coupling the tip 30 or distal end of the device 10 to a fluid source (e.g., a vial inserted within a recess of the handpiece, a manifold system or tower, etc.). In some arrangements, the flow measurement device 60 directly contacts at least a portion of the treatment material or other fluid being transferred through the conduit(s) 50 and/or other portion of the device 10. However, in other embodiments, the flow measurement device 60 does not contact any portion of the treatment material or other fluid being transferred through the conduit(s) 50 and/or other portion of the device 10.

According to some embodiments, measurement of the flowrate of fluid (e.g., treatment fluid, cleaning or rinsing solution, etc.) being transferred to the tip can help confirm the proper function and/or use of the skin treatment device 10. As noted above, for example, a flowmeter 60 can help ensure that an accurate amount of treatment fluid is being delivered to a handpiece. The use of flowmeters 60 can help identify leaks, misuse and/or the like. In some embodiments, the flowmeter 60 comprises an electromechanical flow meter. However, any type of flowmeter 60 can be used, including, without limitation, a positive displacement flow meter, a velocity flow meter (e.g., turbine, paddle wheel, ultrasonic, electromagnetic, vortex-shedding, ultrasonic, optical flow meters, etc.), mass flow meters or any other type of flow meter, as desired or required.

The flowmeter or other flow measurement or flow approximation device or component can be operatively coupled to one or more other components, systems, subsystems and/or other portions of the device 10, including, for instance, batteries or other power sources, data communication lines, controllers, output and/or input devices, control units, memory devices and/or the like, as desired or required. Such connections can be hardwired and/or wireless.

In some embodiments, data collected and processed from one or more flowmeters or other flow measurement components or devices can be used as part of a feedback loop configuration. By way of example, if the flowrate of fluid being delivered to the tip 30 is above or below a particular desired or required level or range, one or more operational parameters of the device (and/or an ancillary system or component to which the device is integrated, e.g., a tower or manifold system) can be modified to achieve the desired or required value or range.

One advantage of placing one or more flowmeters or other flow measurement devices 60 in the actual handpiece 10 is that the measurement or approximation of the flowrate of treatment material or other fluid is in very close proximity to the tip, and thus, the device-skin surface interface. This can provide a more accurate and reliable measurement of fluid and/or other material (e.g., treatment material) that is actually transferred to the skin surface during a treatment procedure.

A handpiece or similar device 10 can include one or more flowmeters or other flow measurement devices or components 60. Such flowmeters or other devices 60 can be placed in series (e.g., within the same fluid path) or in parallel (e.g., in various branches of fluid flow).

### Force Measurement

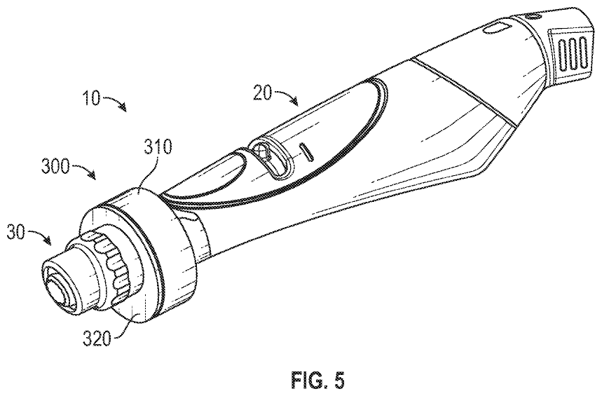

As noted above, in some embodiments, a handpiece 10 can be configured to measure or approximate a force applied by the tip 30 or distal end 12 of the handpiece 10 on a skin surface during a treatment procedure. As illustrated in FIG. 5, the device can comprise a force sensing/detection assembly 300 along its distal end. For example, in the depicted arrangements, the force detection assembly 300 is positioned between the main portion 20 and the tip 30 of the device 10. However, in other arrangements, one or more portions of a force detection assembly 300 can be located at any other location, as desired or required.

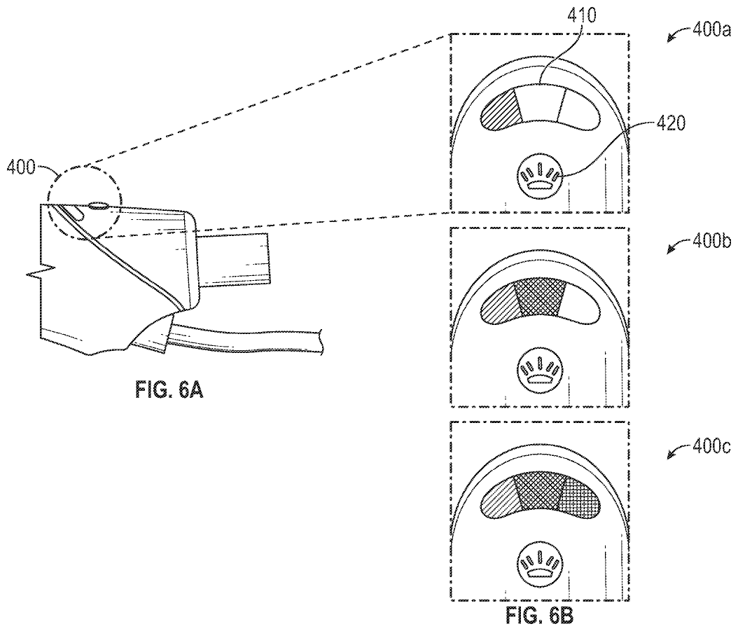

According to some embodiments, force measurements and other data obtained using a force detection assembly 300 can be used to detect, calculate, measure, estimate or approximate force (e.g., actual force, pressure or force over a particular area, other indicia of force whether actual or relative, etc.) being exerted on skin tissue by the handpiece. For any of the embodiments disclosed herein, force detection can include the measurement of positive pressure or force and/or negative pressure or force (e.g., resulting from the application of vacuum or suction). In some arrangements, such measurements or approximation can be presented to the aesthetician or other user of the device 10 in real time. An output of force can be provided to the user on one or more portions of the handpiece itself, such as shown, for example, in FIGS. 6A and 6B.

With reference to FIGS. 6A and 6B, the handpiece or device 10 can include one or more areas that provide an output 400 related to force or pressure applied by the tip or distal end of the device on skin tissue during use. According to some embodiments, such an output 400 can include a visual output that shows relative force or pressure being applied on adjacent skin tissue (e.g., low force or pressure, medium force or pressure, high force or pressure, pressure value, pressure range, etc.). Therefore, in some arrangements, force or pressure is presented to the user (e.g., using one or more outputs 400) in a relative manner. In other embodiments, the handpiece is configured to provide an actual force or pressure measurement or value.

As will be discussed below, a force detection assembly 300 can be incorporated into any handpiece or other handheld device used for skin treatment, including, but not limited to, any of the handpiece and/or other device/system embodiments disclosed herein. For example, although the force detection assembly 300 is illustrated and discussed herein with reference to a skin treatment system that includes fluid delivery and/or suction or vacuum, such features can be incorporated into any device that contacts skin or other tissue of a subject (e.g., even subcutaneous or other internal anatomical portions of the subject).

With continued reference to FIG. 6A, the visual output 400 can be located along the main portion 20 of the handpiece 10. In some embodiments, as shown in FIG. 6A, the output 400 is positioned along a proximal portion of the device 10 (e.g., in an area that facilitates viewing of the output by the user during a treatment procedure). However, the output 400 can be located along any part of the device 10, as desired or required.

The visual output 400 can comprise any desired or required configuration, including lights (e.g., LEDs), numbers, text or other characters, other graphical outputs, etc. For example, FIG. 6B illustrates a 3-level lighted display 410 with a “low force or pressure” reading represented by a first lighted area on the left (e.g., in yellow color), a “medium force or pressure” reading represented by both the first lighted area on the left as well as second lighted area in the middle (e.g., in orange color) and a “high force or pressure” reading by the first and second lighted area on the left and middle, as well as a third lighted area on the right (e.g., in a red color). These varying force/pressure levels are depicted by the examples 400a, 400b, 400c in FIG. 6B.

The exact colors, lighting schemes and/or other details of the display or output 410 can vary. For instance, as noted herein, an actual pressure or force amount or value (e.g., one that is detected, measured, estimated, calculated, etc.) can be displayed. In yet other embodiments, such measurements or representative relative amounts of force or pressure can be provided to a separate device (e.g., a smartphone, a tablet, a computer or other computing device, a separate device, system or component that is operatively coupled to the device, etc.). In yet other embodiments, an actual, relative and/or representative measurement of force or pressure can be audibly provided to the aesthetician. In yet other arrangements, haptic or other feedback can be used to alert the aesthetician or other user that the force or pressure being applied to the skin tissue via the handpiece is too low or too high.

According to some embodiments, the amount of pressure that triggers an output, a warning and/or other alert can be based, at least in part, on the procedure being performed, the treatment materials being delivered to the skin surface, the particular skin condition or problem being targeted and/or other considerations. In some embodiments, the desired amount or range of force or pressure applied by the handpiece on skin tissue is determined by empirical testing (e.g., using data from other treatments for a particular subject, data pertaining to all subject generally, etc.), as desired or required. For instance, the force/pressure settings can be set at a lower scale for a particular subject with sensitive skin that is less tolerant of force from the handpiece.

According to some embodiments, the force detection assembly 300 can be operatively connected to and provide an actual pressure or force amount or value (e.g., one that is detected, measured, estimated, calculated, etc.) to the electronic control system within the handpiece 10. In response to the force related information, the electronic control system (including, e.g., a processor, a memory, a computer-readable medium, an algorithm, etc.) can be configured to modify one or more operational patterns for a procedure according to one or more operational algorithms, including but not limited to: changing the fluid source or selection of container 110A, 110B, 110C, 110D from which the handpiece 10 draws fluid during a procedure; vary the vacuum values (e.g., negative pressure relative to atmospheric) exerted on the subject; vary the flow rate of the fluid provided to the tip 30 of the handpiece 10; toggle activation of a light module 700; obtain imaging data of the surface of the skin of the subject using a camera or other image capture device, which can be stored (e.g., to a memory), processed (e.g., using a processing unit or other computing device) and/or otherwise manipulated to achieve a particular output, result and/or the like; adjusting one or more valves to restrict or otherwise modify flow of a treatment fluid or other fluid or vacuum within or about the handpiece 10, manifold assembly 100, or any system operatively connected to the handpiece 10 or manifold assembly 100; and/or other modifications of operational parameters as disclosed herein or would be modified in response to a force value by one skilled in the art.

With continued reference to FIG. 6B, the output 400 can include one or more visual outputs 420 besides an output representing force or pressure. For example, the output 400 can include an on/off button, and/or a visual indicator showing that force detection is enabled or is working at a baseline level and/or the like.

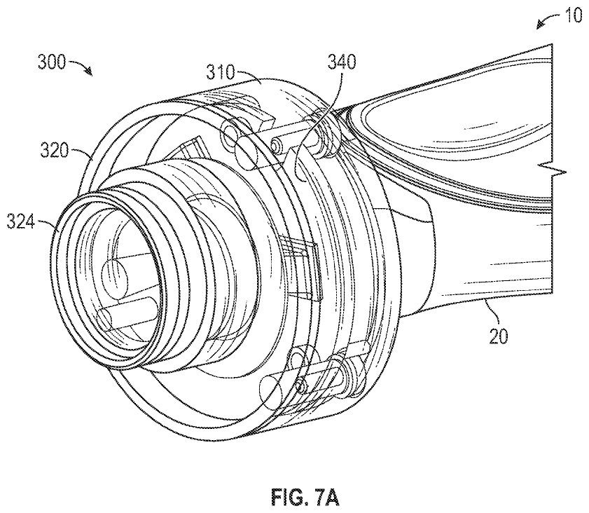

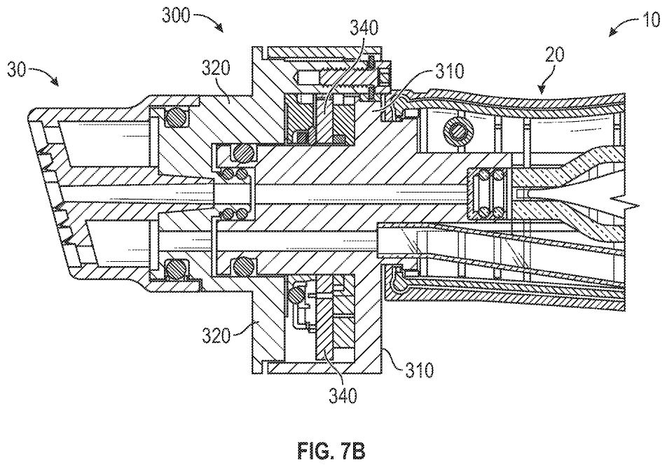

FIGS. 7A and 7B illustrate different views of one embodiment of a force/pressure detection assembly 300. As shown, the assembly 300 can include a proximal or first portion 310 that is secured (e.g., permanently, removably, etc.) to the main portion 20 of the handpiece 10. The assembly 300 can additionally include a distal or second portion 320 with a distal end 324 that is configured to receive (and thus, secure to) a tip 30. As noted herein, however, the assembly 300 can include any other configuration, orientation, location and/or other property or characteristic, as needed or required by a particular application or use.

As best illustrated by the cross-sectional view of FIG. 7B, the distal or second portion 320 of the force/pressure detection assembly 300 is configured to move proximally (e.g., in the direction of the proximal or first portion 310) when the tip is placed along a skin surface and a pressure or force is exerted on said skin surface. As a result, a force is exerted on one or more pressure transducers or other pressure sensors 340 located within and/or on the assembly 300. Thus, the amount of force or pressure exerted by the tip on skin tissue during the execution of a skin treatment procedure can be calculated, estimated and/or other approximated or determined. In some embodiments, calibration of the assembly can be used to ensure that the amount of force or pressure detected is accurately representative of the amount of pressure exerted by the tip (or other portion along the distal end of the device) on skin tissue during use of the device. In some embodiments, a micro pressure sensor can be integrated into the handpiece instead of, or in addition to the pressure sensors 340 present within any embodiment of the force/pressure detection assembly 300 disclosed herein. The micro pressure sensor can assist in measuring or calculating the suction, vacuum pressure, or other operational data as disclosed herein, and can be compatible with or contact (e.g., directly, indirectly, etc.) the treatment fluid or other fluid within or operatively connected to the handpiece 10.

Data measured from the pressure sensor(s) 340 can be provided to another portion of the device for display or processing. For example, in some embodiments, force/pressure data can be provided to a processor (either within the device or outside of the device) for further processing, calibration, manipulation, etc., as desired or required. In one arrangement, the force detection assembly is operatively coupled to an output 400 (e.g., like the output 400 illustrated in FIGS. 6A and 6B and discussed above). In other embodiments, assembly is operatively coupled (e.g., directly or indirectly) to a processor, either In lieu of or in addition to being operatively coupled to an output 400.

With continued reference to FIGS. 7A and 7B, a distal end 324 of the force/pressure detection assembly 300 can be shaped, sized and otherwise configured to receive a removable tip 30. Therefore, in some arrangements, the distal portion 324 of the assembly 300 can include a nozzle shape, an O-ring and/or other seal and/or any other components or features to facilitate receipt and securement of a removable tip 30.

According to some embodiments, the device 10 comprises the necessary electrical and/or other connections to accommodate the force/pressure detection assembly 300. This can include wires, cables, other electrical or electronic components, other wired and/or wireless connection components or devices and/or the like.

The force/pressure detection assembly 300 can be integrated into the handpiece 10 such that the handpiece is provided with the assembly or device 300. However, in other embodiments, a force/pressure detection assembly 300 can be provided as an add-on product that can be added to (e.g., incorporated with, removably or irremovably) any handpiece or similar device, as desired or required. In such embodiments, the assembly 300 can include its own power, connectivity and/or other components to allow it to be integrated into one or more separate handpieces or other devices.

In some arrangements, force or pressure data collected by the detection assembly 300 can be recorded or otherwise saved during a procedure. Such data can be used to confirm that a procedure was conducted properly (e.g., safely, efficaciously, etc.). This can help identify possible issues with the execution of a procedure, can help improve future treatments/iterations of a skin treatment protocol, can help with recordkeeping and/or provide one or more other benefits or advantages.

According to some embodiments, a force/pressure detection assembly can be configured to detect suction or vacuum values (e.g., negative pressure relative to atmospheric), either in addition to and/or in lieu of detecting positive pressure values (e.g., positive pressure relative to atmospheric). In some embodiments, the negative and/or positive pressure value can be displayed or provided (e.g., directly, indirectly, visually, audibly, haptically, combinations thereof, etc.) to the aesthetician or other user. For example, the value can be displayed along one or more potions of the detection assembly and/or another portion of the handpiece. In other embodiments, the negative and/or positive pressure value is provided on a separate device or system that is operatively coupled to the handpiece (e.g., a screen or other output of a tower or console system with a manifold, an output of a separate computing device, etc.). In some embodiments, an indication of relative pressure (e.g., positive, negative) is provided, either in addition to or in lieu of an actual value. For instance, a color scale can be used to indicate relative positive and/or negative pressure. In some embodiments, the system can be configured to provide a warning, terminate a procedure and/or take any other measure in response to certain threshold values being reached (e.g., overpressure, negative or positive, whether it is an instantaneous event, accumulated pressure over time, etc.), as desired or required.

In some embodiments, a cantilevered member with a strain sensor (and/or similar sensor) can be included on, at, along and/or near the tip or other distal portion of a handpiece to facilitate the measurement or approximation of negative and/or positive pressure.

As noted herein, a pressure level or other measurement can be communicated to the user visually, audibly, haptically and/or the like. For instance, the handpiece can be configured to vibrate using one or more haptic components (e.g., positioned on and/or near the handpiece). In other embodiments, a sound is emitted (e.g., by a speaker or other output device located on and/or near the handpiece). In some embodiments, the level of the output (e.g., haptic, audible, etc.) varies according to the intensity of the measurement (e.g., linearly, non-linearly, etc.).

### Tip Detection

A handpiece or other handheld device 10 used in skin treatment procedures can be configured to automatically detect or identify, at least in part, the type of tip 30 that is secured to the handpiece or device 10. In some embodiments, the tips are at least partially translucent and are provided in varying colors. For example, the tips can vary in color based on the pattern of the tip, its intended use (e.g., for which procedures the tips will be used), the skin type of the person undergoing a treatment and/or the like. Tips can include, for instance, blue tips, green tips, yellow tips, red tips, purple tips, pink tips, clear tips, tips of the same general color but of varying brightness, hue, etc. and/or the like.

In some embodiments, a distal portion of the handpiece 10 is configured to automatically detect a specific color of the tip secured to the handpiece 10. Since differences in tip color can be significant or slight, the level of sensitivity of detection can vary, based on the tips being used.

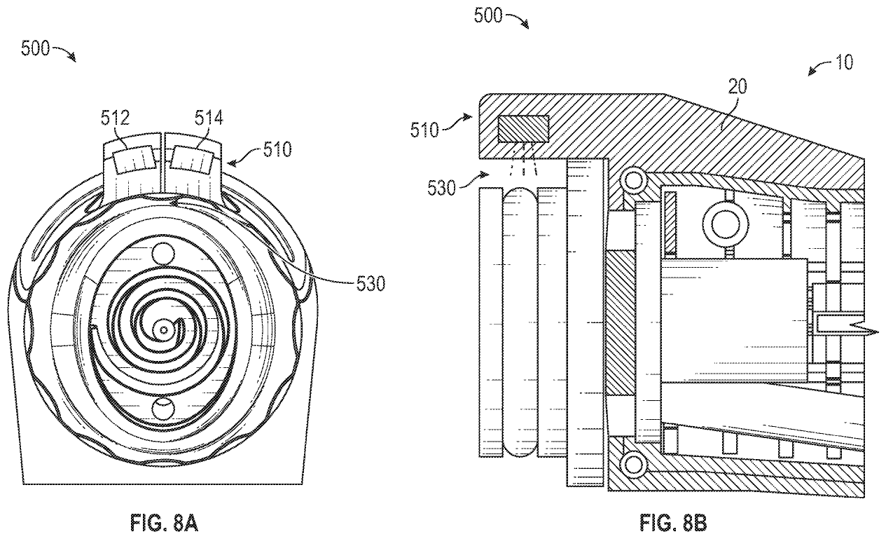

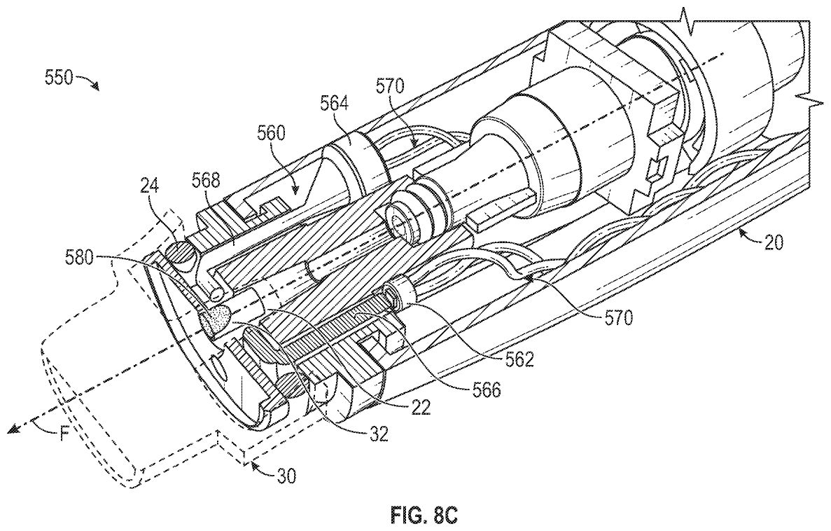

One embodiment of a tip detection assembly 500 is illustrated in FIGS. 8A and 8B. As shown, the detection assembly 500 can be located along the distal end of the device 10 (e.g., along a distal end of the main portion 20 of the device). In some arrangements, the tip detection assembly 500 comprises one or more light sources (e.g., one or more LEDs) 512 and one or more light sensors 514. The light sensor(s) 514 can be designed, located, oriented and/or otherwise configured to receive and detect light emitted by the light source(s) 512. For instance, as illustrated in FIG. 8A, the light source(s) and sensor(s) 512, 514 are adjacent (e.g., immediately, substantially immediately, etc.) to one another (e.g., between 0.1 and 0.5 inches apart). However, in other embodiments, the separation distance between the source(s) and sensor(s) 512, 514 can be greater or less than shown and disclosed herein, as desired or required.

According to some embodiments, the light source 512 comprises a white LED (or other light source) and/or a Red-Green-Blue or RGB LED (or other light source), as desired or required. In some arrangements, light is passed from the light source 512 and through at least a portion of the tip (shown in FIG. 8A) in the tip detection area 530 before it is detected by the light sensor 514. Thus, data collected by the sensor 514 can help identify the color of the tip that is secured to the handpiece. In some embodiments, back scatter techniques are utilized by the sensor, and thus, the tip detection assembly 500, to determine color, allowing the assembly to identify the specific type of tip being used.

For any of the embodiments disclosed herein, including those in connection with tip detection, any sensors can be coupled (e.g., physically, operatively, etc.) to a processor, memory and/or other control components. For example, in some embodiments, data received from any light sensors can be communicated to a processor (e.g., to help determine the tip type, to communicate any conclusions to an output, etc.).

In some embodiments, data and information obtained from the tip detection assembly 500 can be provided to one or more processors (e.g., of the handpiece, of a different system component of an overall skin treatment system, a separate computing device, a network, etc.) to identify the tip that has been secured to the handpiece. Such confirmation can be used to ensure that an appropriate tip has been used for a particular skin treatment procedure. As a result, in some arrangements, one or more operational parameters, alerts and/or other functions of the device and/or overall skin treatment system to which the device is operatively coupled can be adjusted, alerted and/or otherwise activated/deactivated. For example, if the tip that has been secured to the handpiece is not appropriate for a particular procedure, the device or system can provide a corresponding alert to the user (e.g., visually, audibly, haptically, etc.). In other embodiments, the skin treatment device or system can pause a procedure (e.g., until an appropriate tip is secured to the handpiece), terminate a procedure and/or take any other action. In some embodiments, the skin treatment device or system can compare the tip detected and the fluid source or other operational parameter or operational information provided or detected by the system, and propose modifications to or modify one or more treatment parameters. Advantageously, this can increase the efficacy of the detected tip or treatment material, and minimize errors or other discrepancies detected during the course of a treatment procedure.

With continued reference to FIGS. 8A and 8B, the tip detection assembly 500 can include a portion 510 that houses the light source(s) and sensor(s) 512, 514. In some embodiments, the portion 510 comprises a protruding portion that extends distally (e.g., in the direction of the tip). The portion 510 can be positioned such that it can read a portion of the tip 30 in the tip detection area 530 when the tip 30 is connected to the handpiece 10. However, in other embodiments, the portion 510 is aligned with or recessed relative to the distal end of the main portion 20, as desired or required.

In some embodiments, tips include one or more distinguishing visible characteristics, features and/or properties, either in addition to or instead of color and/or other light-based detection. For example, at least a portion of the tip can include markings that can be detected by one or more sensors or detectors. As with other arrangements disclosed herein or equivalents thereof, such detection can be at least partially automatic (e.g., upon securing the tip to the handpiece, passing the tip relative to a detector, etc.) and/or at least partially manual (e.g., involving some input from the user), as desired or required. Markings or other non-color distinguishing features can include, for example and without limitation, dots, stripes, bar codes, other alphanumeric characters, logos, images or other non-alphanumeric markings, etc.

In other embodiments, one or more identification tags (e.g., RFID tags) can be included on and/or within the tip. Corresponding identification tag readers or detectors positioned on or along the handpiece (e.g., a distal end of a main body portion of the handpiece, any other component or portion of the handpiece, etc.) can automatically detect the tag, and thus, identify one or more characteristic or property of the tip secured to the handpiece, such as for example and without limitation, the type of tip, batch number, manufacture place, manufacture date, etc. As discussed herein, data obtained by sensors can be communicated to a processor in order to make a determination or conclusion (e.g., measurement, approximation, etc.), to obtain a measurement or estimation, to provide an output and/or the like.

In other embodiments, an airflow-based technology, magnetic-based technology, optical-based technology and/or any other type of technology can be incorporated into the tip and/or the handpiece to help determine (e.g., automatically) what type of tip has been secured to the handpiece.

According to some embodiments, any of the tip detection assembly configurations disclosed herein can be adapted to detect whether a tip is new or old (e.g., used in a prior procedure, used for a particular amount of time, etc.). One example of such an embodiment is illustrated in FIG. 8C and discussed in greater detail below.

FIG. 8C illustrates a different embodiment of a tip detection assembly 550. The tip detection assembly 550 can be configured to detect whether a tip 30 is able to be used (e.g., the tip is new) or not able to be used (e.g., the tip is used, reused, not new, defective, etc.). Such a configuration can be incorporated into any of the arrangements described herein or equivalents thereof, as desired or required. For example, in one embodiment, a handpiece 20 can include a tip detection assembly that is adapted to detect the type of tip (e.g., as described herein with reference to FIGS. 8A and 8B) and/or detect if the type is new and/or otherwise can be used in a procedure (e.g., as described herein with reference to FIG. 8C).

As with the embodiment depicted in FIGS. 8A and 8B, the detection assembly 550 can be located along the distal end of the device 10 (e.g., along a distal end of the main portion 20 of the device). In some arrangements, the tip detection assembly 550 comprises one or more light sources (e.g., one or more LEDs) 562 and one or more light sensors 564. The light sensor(s) 564 can be designed, located, oriented and/or otherwise configured to receive and detect light emitted by the light source(s) 562. For instance, the light source(s) and sensor(s) 562, 564 can be adjacent to one another. However, in other embodiments, the separation distance between the source(s) and sensor(s) 562, 564 can be greater than shown and disclosed herein. By way of example, as depicted in FIG. 8C, light emitted by the light source 562 can be directed to or near the light sensor or detector 564 using one or more networks of light pipes, channels and/or other passages 560. The use of a light pipe network 560 can advantageously permit the light source or emitter 562 to be positioned further apart (e.g., at a different location, at a different orientation, etc.) relative to the light sensor or detector 564.

With continued reference to FIG. 8C, a light pipe network 560 can be configured to pass light from the light source or light emitter 562 at least partially to and through a portion of the tip 30 that is secured along the distal end of the handpiece 20. In the illustrated embodiment, the light pipe network 560 is configured to deliver light from the light source 562 to and through the stem 32 of the tip 30. In some arrangements, the stem 32 extends proximally along the proximal end of the tip 30 and is sized, shaped and/or otherwise configured to secure into a corresponding opening 22 along a distal end of the handpiece 20. The stem 32 can be configured to removably secure to the opening 22 of the handpiece 20 using a friction fit connection and/or any other type of attachment method or technology (e.g., snap fit connection, threaded connection, etc.), as desired or required. The handpiece 20 can include one or more other components and/or features to facilitate a secure (e.g., leakproof) connection between the handpiece 20 and the tip 30. For example, an O-ring and/or other scaling member 24 can be positioned along the distal end of the handpiece 20 to help eliminate or reduce the likelihood of leaks. Such O-rings or other sealing members or features 24 can be incorporated into any of the arrangements disclosed herein or equivalents thereof.

As illustrated in FIG. 8C, the stem 32 of the tip 30 (and/or any other portion of the tip 30) can include one or more coatings, layers, members, portions and/or features 580 that are configured to at least partially dissolve, dissipate and/or otherwise be removed as fluid is transferred past the stem 32 (and/or other portion of the tip 30 that includes such a coating or portion 580).

According to some embodiments, the partially dissolvable or otherwise removable portion 580 comprises an opaque coating or layer as liquid or other fluid is delivered from the handpiece (e.g., from a vial positioned within the handpiece, from a manifold system in fluid communication with the handpiece, etc.) toward the tip 30 and the skin surface of the subject receiving skin treatment. In FIG. 8C, fluid flow through the handpiece and tip is generally represented by arrow F. Thus, the coating or portion 580 of the tip can be removed from the stem 32 as fluid is delivered through the stem 32 during a procedure. In some embodiments, therefore, a tip 30 that has been used and/or is otherwise not configured to be used in a treatment procedure can be detected by the handpiece 20 and/or the larger skin treatment system. This can provide certain advantages and other benefits to the subject being treated, the aesthetician or other user and/or the overall treatment procedure.

Advantages and benefits of using such configurations can include, by way of example and without limitation, improving hygiene (e.g., by not allowing a used tip to be reused), ensuring the safety to the subject being treated, eliminating or reducing the likelihood of possible mistakes by the aesthetician or other user, preventing the use of tips (e.g., defective or counterfeit tips) that can harm a subject and/or the user, improving efficacy and results (e.g., ensuring that the correct tip(s) are being used in connection with a skin treatment procedure) and/or the like.

In some embodiments, the dissolvable coating, layer or portion 580 is positioned along the entire or substantially the entire interior surface of the tip stem 32. However, in other embodiments, the dissolvable coating or portion 580 is located only along a portion of the interior surface of the tip stem 32, as desired or required. For example, the coating, layer or portion 580 can cover 5% to 95% (e.g., 5 to 95, 5 to 50, 50 to 95, 20 to 80, 30 to 70, 40 to 60, 5 to 10, 10 to 20, 20 to 30, 30 to 40, 40 to 50, 50 to 60, 60 to 70, 70 to 80, 80 to 90, 90 to 95%, percentages between the foregoing values or ranges, etc.) of the interior surface area of the stem 32. In other arrangements, the coating, layer or portion 580 covers 0.1% to 5% or 95% to 100% of the interior surface area of the stem 32.

The coating, layer or other portion 580 that is configured to dissolve and/or otherwise be removed as water, serums and/or other treatment materials or fluids are delivered to and through the tip 30 (e.g., during the execution of a treatment procedure, during use of the tip, during exposure to environmental conditions even if not associated with use of the tip on a handpiece, etc.) can include one or more materials that are configured to dissolve or otherwise separate from the tip 30 (e.g., an interior of the tip's stem 32). The coating 580 can include any material that is safe for the subject who is undergoing a treatment procedure. For example, the coating 580 can include any treatment material that is beneficial to the subject's skin and/or the overall treatment procedure. Materials can include a skin preparation material, a skin cleansing material, a cleansing material, an extraction material (e.g., to facilitate or enhance the removal of blackheads, sebum, other unwanted substances from skin, etc.), a hydrating material, a chemical peel or exfoliation material, a booster material (e.g., peptides, hyaluronic acid, anti-wrinkle material, anti-acne material, etc.) and/or the like, as desired or required.

In some embodiments, the coating, layer or other dissolvable or removable portion 580 of the stem 32 (and/or other portion of the tip 30) can include a material that will be delivered to the skin surface of the subject during the particular treatment procedure for which the tip 30 will be used. The coating, layer or other dissolvable portion 580 is safe to the user's skin and/or will not impact (e.g., at least will not materially impact or alter) the corresponding skin treatment procedure.

In some embodiments, the material(s) included in the coating 580 can be different than the materials (e.g., serums, boosters, etc.) that are delivered to the subject's skin surface during the treatment procedure. Regardless of what material(s) is/are used for the coating 580, the coating 580 is preferably safe to the subject and the aesthetician (or other user or individual who may come in contact with the tip) and/or will not have a negative impact or adverse effect (e.g., at least not a detectable and/or material impact of effect) on the subject, the user and/or the overall treatment procedure in connection with the treatment in which the tip will be used.

According to some embodiments, the coating 580 is at least partially opaque so that it can be detected by the light sensor 564 during use. By way of example and without limitation, the opacity of the coating 580 before the tip 30 is used (e.g., before any fluid has passed through the stem 32 or other portion of the tip 30 that includes the coating 580) is 10% to 100% (e.g., 10 to 100, 10 to 30, 30 to 50, 50 to 100, 50 to 90, 10 to 90, 40 to 60, 50 to 70, 10 to 40, 70 to 100, 70 to 90%, values between the foregoing values or ranges, etc.). In other arrangements, the opacity of the coating 580 prior to use is less than 10% (e.g., 0.1 to 10, 1 to 10, 5 to 10, 0.1 to 5%, values between the foregoing values or ranges, etc.), as desired or required.

The handpiece 20 (and/or the overall skin treatment system of which the handpiece is a part) can be configured to detect whether a tip has been previously used and/or whether a tip should be used in another procedure based on a measurement of light detected by the sensor or detector 564. For example, the handpiece 20 and/or the overall skin treatment system can be adapted to determine that a particular tip 20 should not be used for a skin treatment procedure because the opacity of the coating or other portion 580 of the stem 32 (and/or other portion of the tip 30) is below a particular threshold level. In some embodiments, such a threshold level is fixed. The threshold level can be determined or set by the manufacturer of the handpiece and/or the tip. However, in other arrangements, the threshold level can be customized and/or adjusted (e.g., by the manufacturer, the user, etc.), as desired or required. By way of example and without limitation, the threshold level of opacity that signals that a tip has been previously used (and/or at least should not be used again) can be a percentage drop of opacity relative to the original (e.g., manufactured) opacity of the coating or other opaque portion 580. For instance, a drop in opacity of 5% to 50% (e.g., 5 to 50, 5 to 10, 10 to 20, 20 to 30, 30 to 40, 40 to 50%, values between the foregoing ranges or values, etc.) can be indicative that the tip should not be used. In other arrangements, a drop in opacity of less than 5% or greater than 50% can be used as such a triggering or threshold level.

According to some embodiments, the opacity of the coating 580 is configured to drop below the threshold level when only a relatively small volume of fluid has been passed through the tip (e.g., the stem 32 that includes the coating 580). For example, the opacity can be configured to drop below the threshold when 1 to 20 milliliters (e.g., 1 to 2, 2 to 5, 5 to 10, 10 to 20 ml, volumes between the foregoing ranges, etc.) has passed through the stem or other relevant portion of the tip 30. In other arrangements, the opacity can be configured to drop below the threshold when less than 1 ml (e.g., 0.1 to 0.2, 0.2 to 0.5, 0.5 to 1 ml, volumes between the foregoing ranges, etc.) or more than 20 ml (e.g., 20 to 30, 30 to 50, 50 to 100 ml, volumes between the foregoing ranges, more than 100 ml, etc.) has passed through the stem or other relevant portion of the tip 30, as desired or required.

In some embodiments, the passage of time can cause the opacity of the coating or other dissolvable or removable portion 580 of the tip to decrease or otherwise change. For example, the tip 30 can include a coating or other portion 580 that reacts to ambient conditions to reduce the opacity of the coating or portion 580 over time, even if the tip 30 is not used in a treatment procedure. This can help ensure that the shelf life of a tip 30 is not undesirably exceeded. In some arrangements, for example, the opacity of the coating 580 can be configured to decrease (e.g., below a threshold level) in a few months or years (e.g., within 1 month, within 2 months, 1 to 3 months, 3 months to 6 months, 6 months to 1 year, 1 year to 2 years, greater than 2 years, time periods between the foregoing ranges, etc.) upon being applied to a tip 30 or portion 580 thereof. In one embodiment, such a time period coincides, at least generally or substantially, with the shelf life of the tip 30. In such configurations, the opacity coating 580 can be adapted to decrease as a result of oxidation and/or other environmental occurrences or conditions (e.g., in the presence of air, humidity, ambient light, temperature fluctuations and/or other ambient conditions), as desired or required.

For a tip 30, the thickness of the opaque coating 580 can be uniform, substantially uniform and/or non-uniform or substantially non-uniform. In some embodiments, the thickness of the coating 580 is 0.01 to 2 mm (e.g., 0.01 to 1, 0.1 to 1, 0.01 to 2, 0.1 to 2, 1 to 2 mm, values between the foregoing ranges, etc.). In other arrangements, the thickness of the coating 580 can be less than 0.01 mm or greater than 2 mm, as desired or required. For example, the thickness of the coating 580 can be increased if the material(s) used in the coating 580 is/are more susceptible to dissolving. Similarly, the thickness of the coating 580 can be reduced if the material(s) used in the coating 580 is/are less susceptible to dissolving.

The opaque coating 580 can be added to the tip 30 (e.g., an interior portion or region of the tip stem 32) at the time of manufacture of the tip 30. Alternatively, the coating 580 can be added following the tip's manufacture. The coating 580 can be deposited onto and/or otherwise applied to the tip (e.g., along the stem's interior) using any method or technology, such as, for example and without limitation, spraying, casting, dipping, via a roller, brush or similar device, layer-by-layer deposition, optical deposition, other deposition techniques and/or the like.

In alternative embodiments, the handpiece 20 and/or the overall skin treatment system of which the handpiece is a part is configured to prevent or otherwise limit use of a tip when the opacity of a portion of the tip (e.g., analogous of the coating 580) is above a particular threshold. For example, the tip can include a portion that reacts to fluid passing through the tip 30 (e.g., toward a skin surface being treated) and becomes more opaque. Although this is the opposite of the embodiments discussed above, the general concept of using a light source and corresponding light sensor or detector to determine if a tip should be used is just as pertinent. In such arrangements, the opacity of the relevant tip portion 580 can increase as a result of a physical (e.g., presence of fluid, temperature change, etc.) and/or chemical occurrence (e.g., the presence of a non-clear serum or fluid, a chemical reaction between a serum or fluid and the portion 580 and/or the like).

According to some embodiments, if the light sensor or detector 564 detects an opacity of the portion 580 that is below a threshold level (or above a threshold level in an alternative configuration), the handpiece 20, the overall skin treatment system of which the handpiece is a part and/or any other components, device or system that is associated with the handpiece and/or skin treatment system can be configured to take one or more actions. For example, a processor of and/or associated with the handpiece or the overall skin treatment system can terminate or prevent (and/or otherwise limit one or more aspects of an operation of) a skin treatment procedure from beginning or continuing if a determination is made that the tip 30 secured to the distal end of the handpiece 20 is used or may have already been used (e.g., based on an opacity reading). In some embodiments, the handpiece and/or the skin treatment system is configured to alert the user that the tip is or may have already been used, either in addition to or in lieu of terminating or preventing a treatment procedure from commencing or continuing. The handpiece, any other portion of the skin treatment system and/or any other component, device or system operatively coupled to the handpiece or skin treatment system can be adapted to provide an audible, visual, haptic and/or other type of alert to the user. In some embodiments, the device and associated system disclosed herein are configured to generate, send and/or otherwise transmit or handle an electronic communication (e.g., remote communication, non-local communication, email, SMS or other message, etc.) if a corresponding determination is made by the device or system regarding the tip.

With continued reference to FIG. 8C, the handpiece can include wiring or other electrical connections 570 and/or any other components or features to electrically couple the components of the tip detection system to other portions of the handpiece and/or the overall skin treatment system. The handpiece 20 can also include one or more other components, as desired or required, such as, for example and without limitation, a power source, sensors, processors, controllers, etc.

The embodiment depicted in FIG. 8C comprises a light piping system 560 to deliver light from the light source or emitter 562 to the light sensor or detector 564. Such a light piping system or network can include one or more light pipes 566, 568 and/or other members, fittings or portions (e.g., elbows, bends, etc.) to pass light from the light source 562, through the opaque portion 580 and to the light sensor or detector 564. However, in other embodiments, the handpiece can include a light emitter or source 562 and a corresponding light sensor or detector 564 without the use of light pipes or other light delivery network (e.g., similar to the system illustrated in FIGS. 8A and 8B), as desired or required. Further, a handpiece that is configured to detect the type of tip that is secured to the handpiece, such as the one depicted in FIGS. 8A and 8B, can include a light piping network similar to the one illustrated in FIG. 8C. The light pipe network or system 560 is simply used to position the light emitter or source at a location that is further apart from (e.g., not immediately adjacent to) the light sensor or detector. Further, in some embodiments, a handpiece can be configured to determine both the type of tip that is secured to the distal end of the handpiece (e.g., as discussed herein with reference to FIGS. 8A and 8B) and to determine if such a tip is used or may have already been used (e.g., as discussed herein with reference to FIG. 8C) using the same light source and light sensor or detector.

### Vial/Container Identification

According to some embodiments, a handpiece or device 10 is configured to automatically detect identification information provided on a vial or other container positioned within a recess of the handpiece or otherwise secured to the handpiece. For instance, the handpiece 10 can include an identification detection assembly 600 that is configured to identify one or more RFID tags or other identifiers (e.g., other electromagnetic identifiers, graphic or other visual identifiers such as QR codes, barcodes, alphanumeric codes, etc.) secured to or otherwise associated with a serum or other treatment fluid container. The tags or other identifiers can help ensure that the correct serums and other treatment serums are being utilized for a specific skin procedure or other protocol.

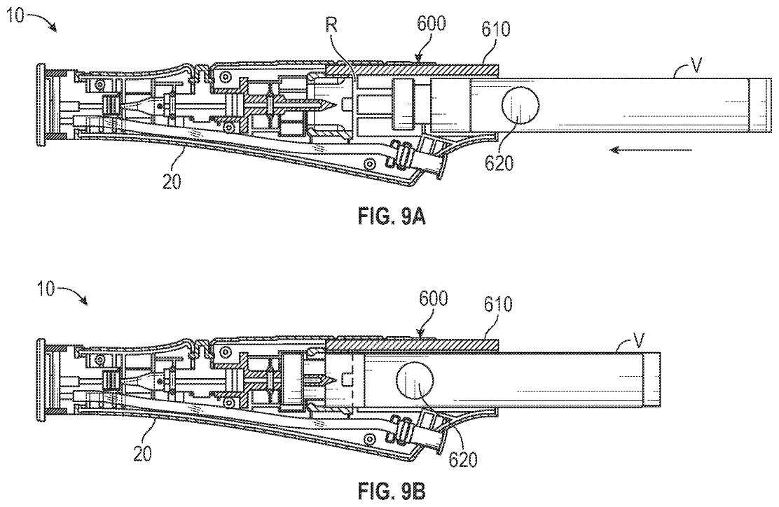

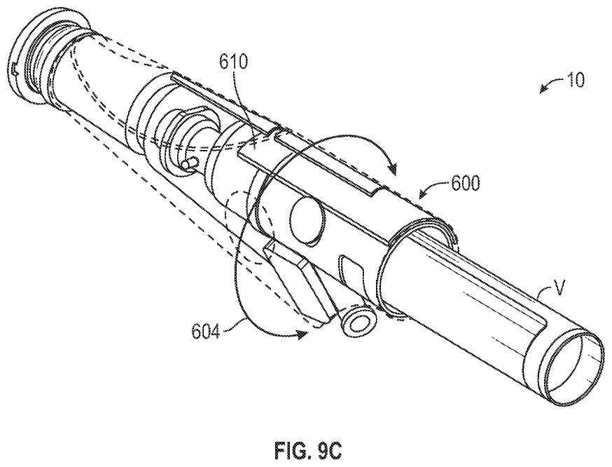

FIGS. 9A to 9C illustrates one embodiment of a handpiece 10 comprising a recess or other opening R that is sized, shaped and otherwise configured to receive a vial, cartridge or other treatment container V. As shown in the depicted arrangements, the vial or other container V can include one or more automatic identification tags 620, such as, for example, a RFID tag or chip, a barcode, etc. In embodiments where the tag 620 is positioned along the curved outer surface of the vial, the tag 620 can be flexible. In other embodiments, the tag 620 is rigid, semi-rigid or only partially flexible, as desired or required. The identification tag 620 is located along the curved outer cylindrical surface of the vial. However, the tag can be positioned along any other portion of the vial, such as, for example, the cap or upper portion, the bottom surface and/or the like.

Regardless of their exact type, size, shape, other material properties, methods of attachment to the vial or other container, configuration, location, etc., such tags 620 can be used to advantageously store information regarding the specific bottle, vial or other container V on which they are positioned or to which they are otherwise secured. For example, the tag 620 can include information regarding the contents of the container, expiration date, manufacturing date, size, lot number, skin procedure with which the contents are intended to be used, other limitations or restrictions on use (e.g., counter-indications, adverse effects, other fluids with which the contents should not be combined, etc.).

The RFID or other identification tag can be read or otherwise detected (e.g., automatically, manually, etc.) by one or more readers or detectors 610 of the identification detection assembly 600. For example, in some embodiments, such a reader 610 can be placed at or near the recess R of the handpiece 10, immediately adjacent where the vial or other container V is secured to the recess R. Accordingly, the RFID or other type of reader 610 of the assembly 600 can detect and identify the RFID tag of the vial or other container.

In some arrangements, placement of a vial within such a recess or other opening of the handpiece 10 (and/or removal of a vial from handpiece) can automatically allow the reader 610 to obtain information regarding the vial.

In circumstances where the detected identifier is inconsistent with the proper, predicted, safe, appropriate and/or approved operation of the device, the device (or the overall system with which the device is associated) can be configured to prevent fluid from that vial or container from being used (e.g., by terminating the vacuum source, by maintaining a solenoid valve or other valve in the closed position, by disabling the controllers of the handpiece, etc.). In some embodiments, n circumstances where the detected identifier is inconsistent with the proper, predicted, safe, appropriate and/or approved operation of the device, the device (or the overall system with which the device is associated) can be configured to propose modifications to or modify one or more treatment parameters. Advantageously, this can increase the efficacy of the detected tip or treatment material, and reduce the likelihood errors or other discrepancies detected during the course of a treatment procedure.

The use of the RFID or other identification tags on vials and/or other containers configured to be used with a handpiece and/or an overall skin of the system can provide one or more other advantages or benefits. The collection of data regarding use of and/or related to the corresponding container (e.g., vial) can be gathered or otherwise collected to generate reports for billing, reordering and/or other purposes. In some embodiments, the number of times that a vial or other container can be removed and reinserted within a handpiece can be limited (e.g., 1, 2, 3, 4, etc.), as desired or required. For example, such limits can help prevent or reduce the likelihood of contamination of the fluid. In some embodiments, the automatic identification of the vial or other container being secured to the handpiece can allow the device and/or related system to determine if a rinse, flush and/or other steps or actions are required before the fluid from that vial or other container can be used and/or any other action can be taken in connection with use of the device.

According to some embodiments, the use of RFID or other identification tags can facilitate the execution of a particular skin treatment protocol. For instance, a procedure may require a variety of different treatment materials and/or other fluids. Thus, different vials may be necessary to carry out any one of a number of various skin treatment procedures. For example, a treatment sequence can be configured for use in procedures for periodic or normal microdermabrasion treatment, anti-aging, anti-acne, skin lightening, oily skin treatment and/or the like. Each of the sequences, protocols or modes can include the delivery of one, two or more various serums and/or other fluids that are housed in different vials. Accordingly, a handpiece 10 that includes an identification detection assembly 600 as disclosed herein can ensure that the proper vial is secured to the handpiece before a particular procedure can begin or continue.

With continued reference to FIGS. 9A to 9C, the identification detection assembly 600 can include a flexible RFID or other type of identification tag antenna or reader 610. In some embodiments, such an antenna or reader 610 extends only partially around the recess or opening R into which the vial or other container V will be positioned. For example, in some arrangements, the reader 610 extends 0 to 120 degrees (e.g., 0 to 120, 0 to 90, 0 to 60, 0 to 45, 0 to 30, 10 to 120, 10 to 120, 10 to 90, 20 to 80, 30 to 70, 20 to 100 degrees, values between the foregoing values and ranges, etc.) around the radially around the recess or opening R. In other embodiments, the

In some embodiments, the length of the antenna or reader 610 is 0.1 to 6 inches (e.g., 0.1 to 0.5, 0.5 to 1, 1 to 2, 2 to 3, 3 to 4, 4 to 5, 5 to 6, 0.1 to 2, 0.1 to 3, 1 to 3, 2 to 5 inches, values between the foregoing, etc.). The reader 610 can be configured to automatically detect the RFID or other identification tag 620 of the vial before the vial V has been fully inserted within the recess or other opening R of the handpiece 10. For example, the reader 610 can be configured to read the identification tag (e.g., RFID tag) 620 of the vial V when the vial V was positioned only 0 to 75% (0 to 75, 0 to 10, 0 to 20, 0 to 30, 0 to 40, 0 to 50, 0 to 60, 10 to 70, 20 to 60%, values between the foregoing values, etc.) of the full insertion length of the recess or other opening R of the handpiece 10.

According to some embodiments, the reader 610 is configured to detect the RFID or other identification tag 620 of a vial or other container V regardless of the rotational orientation of the vial or container. Such a rotational-neutral feature is generally represented by arrow 604 in FIG. 9C.

In some embodiments, a vial or other container can include one or more distinguishing visible characteristics, features and/or properties. For example, at least a portion of the vial (e.g., a label affixed thereto, the glass or other outer surface, a cap or other sealing member, etc.) can include markings that can be detected by one or more sensors or detectors. As with other arrangements disclosed herein or equivalents thereof, such detection can be automatic (e.g., upon securing the vial or similar container within a recess or other opening of the handpiece) or manual (or partly manual, e.g., involving some input from the user), as desired or required. Markings or other non-color distinguishing features can include, for example and without limitation, dots, stripes, bar codes, other alphanumeric characters, logos, images or other non-alphanumeric markings, etc.

In some embodiments, the handpiece is configured to determine or estimate how much fluid remains within the vial or other container that is secured to the handpiece. In some embodiments, the vial includes a label that is adapted to allow the handpiece and/or the user to automatically or manually assess how much fluid remains in the vial or other container. For instance, the vial or other container can include a component (e.g., the cylindrical portion of the vial, the label, etc.) that is at least partially transparent, translucent and/or otherwise visible. In some arrangements, the label can include one or more open sections (e.g., strips, omissions, etc.) to allow for viewing of at least a portion of the internal contents of the vial or other container. In some embodiments, such sections can allow the user itself to determine how much fluid remains in the vial (and relatedly, how much fluid has been used). In other embodiments, one or more sensors (e.g., optical sensors) can be included in the handpiece to allow the system to automatically determine (e.g., in real-time) how much fluid remains in the vial or other container. In some arrangements, the handpiece is configured to provide illumination to the vial to facilitate the manual and/or automatic determination of how much fluid remains in the vial or other container. In some embodiments, the illumination source comprises one or more LEDs and/or other light sources positioned along one or more portions of the handpiece (e.g., at or near the recess that is configured to receive a vial or other container, another location of the handpiece, etc.), as desired or required.

### Light Emission & Imaging/Detection

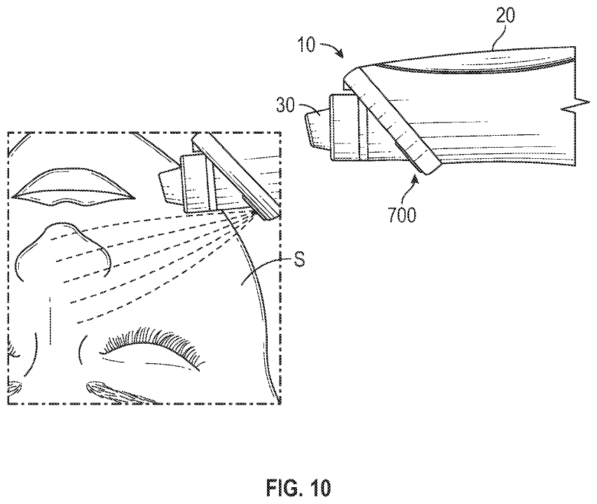

For any of the embodiments disclosed herein or equivalents thereof, a handpiece or device 10 can include one or more integrated features 700. One configuration of such integration 700 is generally depicted in FIG. 10. As shown, a distal end of the handpiece 10 can include a module 700 that is configured to perform one or more functions, such as, for example and without limitation, emit light (e.g., in the direction of skin tissue, distally relative to the handpiece, etc.) at one or more wavelengths. Further, either in addition to or in lieu of light emitters, the handpiece can include one or more of the following: sensors, camera and/or other imaging components, devices, systems or technologies, etc. (e.g., to capture images, or measure/detect one or more parameters, etc.).

In some embodiments, the module 700 is fixed to the handpiece 10 along the distal end of the handpiece, e.g., generally between the main portion 20 and the tip or the distal most end of the handpiece 30. However, in other arrangements, as illustrated in FIG. 10, the module 700 is removably positioned along a portion (e.g., a distal portion) of the handpiece 10, as desired or required. Further, in the configuration of FIG. 10, the module 700 is angled relative to both the longitudinal axis of the handpiece and a plane perpendicular or substantially perpendicular to the longitudinal axis of the handpiece.

As illustrated in FIG. 10, the module 700 can be configured to emit light in the direction of a subject's skin tissue during use (e.g., while a treatment procedure is in progress). Thus, light emitted from the module 700 can be predictably and conveniently directed toward the subject's skin surface S.

The module 700 can be configured to permit a user to modify one or more aspects of the light that is directed toward a targeted skin surface S. For example, a user can be permitted to modify the color, wavelength, brightness, intensity and/or any other property or aspect of the light being emitted from the module 700. Such modifications to operational parameters of the module 700 can be made by the user using one or more controllers. Such controllers (not shown) can be positioned on the handpiece 10, on a separate device or system (e.g., larger skin treatment system, ancillary skin treatment device, a separate computing device, a smartphone or tablet, etc.) and/or the like. In other embodiments, such changes can be made through audible instructions, through gesture control and/or using any other technology.

Accordingly, for any of the embodiments disclosed herein, a skin treatment system and/or the method being performed using a skin treatment system can incorporate one or more light treatment steps or evaluations/assessments using light emission and detection.

The light emitted by the module 700 can be provided by one or more light emitting diodes (LEDs). As noted above, the color (e.g., blue light, red light, etc.), intensity, duration and/or one or more other features related to the LEDs or other light source can be adjustable to accomplish a particular goal or purpose. For example, light emission and detection (e.g., using light sources, sensors, cameras, etc.) can facilitate assessment of a subject's skin and the health of said skin. Further, such assessment can be tracked over time to understand and improve the progress being made by a subject on a longer-term basis. Such a time basis can include several days, weeks, months and even years. In other embodiments, light can be used for, at least in part, therapy, such as, for instance, assisting in improving the result of treating skin for acne, aging, inflammation, etc.

In some embodiments, blue light is used to detect sebum, porphyrins and/or other materials, substances or conditions indicative or suggestive of an infection (e.g., a bacterial infection) or other skin ailment or condition. In some arrangements, a wavelength of the light is 300 to 400 nanometers or nm (e.g., 365 nm, 360 to 370 nm, 350 to 380 nm, 320 to 390 nm, 300 to 400 nm, values between the foregoing values or ranges, etc.). Such blue light delivery can be helpful for skin where sebum, bacteria and/or the like are an issue. For example, in some embodiments, sebum fluoresces (e.g., in yellow or a yellowish color) or is otherwise more easily identified when light (e.g., blue light) is applied to the skin. Accordingly, an aesthetician or other user or operator can easily identify portions of the skin that need treatment. In other embodiments, light can include one or more other wavelengths (e.g., outside the spectrum indicated above, smaller and/or greater wavelengths) to identify one or more other target substances and/or conditions. For example, light can be delivered at 390 to 410 nm (e.g., 405 nm, 390 to 410 nm, 395 to 400, 400 to 405, 402 to 407 nm, values between the foregoing ranges and values, etc.).

In some embodiments, control of the light source can be integrated with other control features of the handpiece 10 and/or a larger skin treatment system of which the handpiece may be only one component. For example, as discussed, one or more properties of the LEDs or other type(s) of light of the light source can be adjusted (e.g., manually, automatically, etc.) using controllers, a main touchscreen of a related tower or console system, using a separate device (e.g., a PC operatively coupled to the system, a smart device, etc.), as desired or required.

According to some arrangements, the light module 700 comprises both light emitting and light sensing capabilities. Such configurations can permit the light module 700 to direct light on a subject's skin and generate an assessment image or “map” of a targeted skin surface. In some embodiments, such a map can identify areas that arc damaged (e.g., and thus, should be avoided for treatment or at least should be treated to a lesser degree than adjacent areas), are in need of further or specific treatment (e.g., identify sebum, oils, blackheads for additional treatment steps) and/or the like.

In some embodiments, a camera or other image capture device can be included in the module 700. Such a camera or other image capture device can be used to obtain imaging data of the subject's skin. Any data obtained from the camera and/or other light sensors of the module 700 can be stored (e.g., to a memory), processed (e.g., using a processing unit or other computing device) and/or otherwise manipulated to achieve a particular output, result and/or the like. In some arrangements, photographs, skin maps and/or other imaging samples are obtained, saved and/or processed during each visit to the aesthetician or other professional. Therefore, in such embodiments, the progress of a skin treatment regimen, the overall health of the subject skin, recommended changes to a skin treatment protocol and/or other information that can assist with assessing a subject skin and/or treating it can be enhanced.

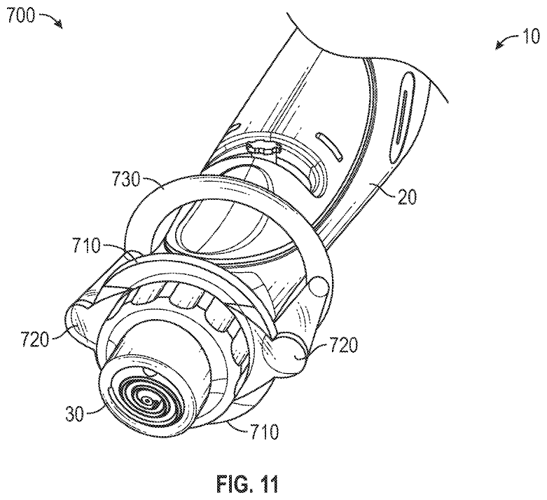

FIG. 11 illustrates one embodiment of a light module 700 that is positioned along a distal end of a handpiece 10. As shown, the module 700 can be generally positioned between the main portion 20 of the handpiece 10 and the tip 30 secured along the distal end of the main portion 20. In some arrangements, the module 700 is fixed to the handpiece 10 such that it is not intended to be easily removed. For example, with exceptions for professional repair or maintenance, the light module 700 may be configured to remain secured to the handpiece 10. In other arrangements, however, the module 700 is designed and otherwise configured to be removed from the main body 20 and/or other portion of the handpiece 10. In yet other arrangements, the module 700 can be secured to or incorporated into the tip 30 and/or may be configured to be separated (e.g., for removal, replacement, maintenance, repair, etc.) from both the main body 20 (or other portion of the handpiece 10) and the tip 30, as desired or required.

With continued reference to FIG. 11, the light module 700 can include one or more LEDs or other sources of light 710. In the depicted embodiment, the light source 710 includes one or more LEDs that have an annular or circular orientation. The LEDs or other light source 710 can extend radially around the entire longitudinal axis of the tip and handpiece, as illustrated in FIG. 11. However, in other embodiments, the light source 710 extends only partially around the longitudinal axis of the tip and handpiece, as desired or required. For example, the LED or other light source 710 can extend 5 to 350 degrees around the axis (e.g., 5 to 45, 45 to 90, 90 to 120, 120 to 180, 180 to 350, 90 to 270, 45 to 270 degrees, angles between the foregoing values and ranges, etc.).

With continued reference to FIG. 11, the light module 700 can further include one or more additional light sources and/or custom optic components 720. For example, in the illustrated arrangement, the module 700 comprises two custom light sources 720 on opposite or substantially opposite ends of the tip 30 and handpiece 20. Such light sources can include, for instance, specific types of light (e.g., blue light) that can be used for imaging the skin surface, obtaining data related to the skin, assisting with treatment and/or the like.

In some arrangements, one or more of the light sources 710, 720 of a light module 700 are configured to emit ultraviolet (UV) light, blue light and/or any other type of light. As discussed above, the light sources 710, 720 can be configured to permit a user to modify one or more aspects of the light emitted therefrom. For example, the color, wavelength, intensity, brightness and/or any other operational parameter can be modified by a user or the device/system itself. Such changes can be performed manually (e.g., by the aesthetician, other professional, other user, etc.) or automatically (e.g., by the device, system, etc.).

A light module 700 can include one or more additional components 730, such as, for instance, a printed circuit board (PCB), a printed wiring board, a chip, a memory, wiring, sensors, other electronic or electrical components and/or the like, as desired or required. As noted above, the light module 700 can be separated from the handpiece 10 and/or the tip 30 for removal, replacement, maintenance, repair and/or any other function or purpose. In some embodiments, the module is sized, shaped and otherwise configured for use with existing handpieces or devices. As such, the module 700 can be provided as an aftermarket product. In alternative configurations, however, as noted above, the module 700 can be integrated into new handpieces or devices (e.g., as a fixed or removable component to the handpiece 10, tip 30 and/or the like).

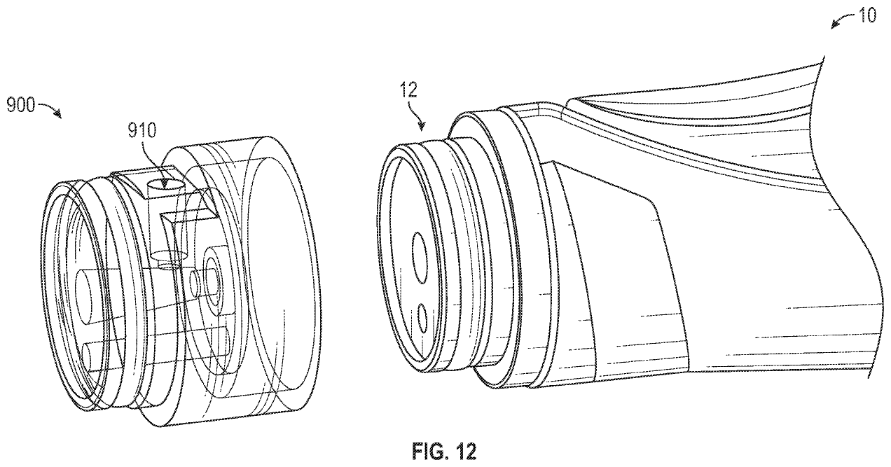

In some embodiments, a handpiece 10 is configured to measure a temperature on, along or near the skin surface of a subject. FIG. 12 illustrates one embodiment of a temperature detection module or assembly 900 that is positioned at or near the distal end of a handpiece or other handheld device 10. As shown, the module 900 can include one or more temperature sensors 910. In some embodiments, the sensor comprises a thermistor. The thermistor or other sensor 910 can be hermetically sealed or otherwise at least partially encapsulated or enclosed. Such configurations can help protect the integrity of the sensors and/or provide one or more additional benefits or advantages (e.g., extending the life of the sensor, preventing damage to the sensor, etc.).

With continued reference to FIG. 12, the temperature detection module 900 can be (or can include) an adapter that is configured to be positioned between a main portion of the handpiece or device 10 and a tip (not shown). Thus, in some embodiments, a distal end or portion of the module 900 is shaped, sized and otherwise configured to receive a tip. In some embodiments, the module is configured to be removably secured to the main portion or other part of the handpiece or device. However, in other arrangements, the module 900 is fixed to the handpiece or device, as desired or required. In other embodiments, the module is configured to be removably or fixedly secured to a tip. While in yet other arrangements, the features of the module (e.g., the sensor(s) 910, other components, etc.) can be incorporated into a tip. For example, in some such embodiments, the tip and the module are joined in a single unitary or monolithic component.

Regardless of its exact design and configuration, a temperature detection module 900 can be configured to detect the temperature of a skin surface, the temperature of fluid passing by or near the module and/or any other temperature measurements obtained by the one or more sensors (e.g., thermistors) of the module 900. In some embodiments, temperature measurements obtained by the module 900 can be used as part of a control scheme for the device. In some embodiments, the temperature detection module 900 includes and/or is operatively coupled to a processor unit (e.g., a processor, a memory, etc.) that receives information from one or more sensors and provides (e.g., calculates, determines, communicates, etc.) one or more outputs (e.g., visually, audibly, haptically, etc.).

For any of the embodiments disclosed herein, a handpiece or device can include the necessary control functions to permit an aesthetician, other professional or other user of the handpiece or device to obtain information (e.g., force/pressure measurement, imaging or mapping, temperature measurements, etc.) and/or make adjustments to the operation of the handpiece or device during use (e.g., increase/decrease flowrate, activate light delivery, adjust temperature of fluid/skin surface, etc.). Such control can be accomplished with one or more controllers (e.g., buttons, switches, dials, touchscreens, etc.), audible commands, gesture control and/or the like. Any controllers or input devices or technologies can be incorporated into the handpiece or device itself, and/or into a separate system or device (e.g., a console, tower or another component of a skin treatment system, a computer, tablet, smartphone or other separate computing device, etc.).

Although several embodiments and examples are disclosed herein, the present application extends beyond the specifically disclosed embodiments to other alternative embodiments and/or uses of the inventions and modifications and equivalents thereof. It is also contemplated that various combinations or subcombinations of the specific features and aspects of the embodiments may be made and still fall within the scope of the inventions. Accordingly, it should be understood that various features and aspects of the disclosed embodiments can be combined with or substituted for one another to form varying modes of the disclosed inventions. Thus, it is intended that the scope of the present inventions disclosed herein should not be limited by the particular disclosed embodiments described above but should be determined only by a fair reading of the claims that follow.

While the inventions are susceptible to various modifications, and alternative forms, specific examples thereof have been shown in the drawings and are herein described in detail. It should be understood, however, that the inventions are not to be limited to the particular forms or methods disclosed, but, to the contrary, the inventions are to cover all modifications, equivalents, and alternatives falling within the spirit and scope of the various embodiments described and the appended claims. Any methods disclosed herein need not be performed in the order recited. The methods summarized above and set forth in further detail below describe certain actions taken by a practitioner; however, it should be understood that they can also include the instruction of those actions by another party. The methods summarized above and set forth in further detail below describe certain actions taken by a user (e.g., a professional in some instances); however, it should be understood that they can also include the instruction of those actions by another party. Thus, actions such as “moving a handpiece” or “delivering a fluid” include “instructing moving a handpiece” and “instructing delivering a fluid.” The ranges disclosed herein also encompass any and all overlap, sub-ranges, and combinations thereof. Language such as “up to,” “at least,” “greater than,” “less than,” “between,” and the like includes the number recited. Numbers proceeded by a term such as “about” or “approximately” include the recited numbers. For example, “about 10 mm” includes “10 mm.” Terms or phrases preceded by a term such as “substantially” include the recited term or phrase. For example, “substantially parallel” includes “parallel.”

What is claimed is:

1. A skin treatment device comprising:
a handpiece comprising a distal end, the handpiece configured to receive a tip along the distal end; and
a tip detection assembly configured to automatically detect a property of the tip secured to the handpiece;
wherein the tip detection assembly comprises at least one light emitter and at least one light detector, wherein light emitted from the at least one light emitter is configured to be detected by the at least one light detector, a light path existing between the at least one light emitter and the at least one light detector; and
wherein at least a portion of the tip is configured to be within the light path of the tip detection assembly when the tip is secured to the handpiece.

2. The device of claim 1, wherein the tip detection assembly is configured to detect whether the tip is or may be new.

3. The device of claim 1 or 2, wherein the tip detection assembly is configured to detect whether the tip should be used in a skin treatment procedure.

4. A device according to any one of claims 1 to 3, wherein the tip detection assembly is configured to detect the type of tip that is secured to the handpiece.

5. The device of claim 4, wherein the type of tip is based, at least in part, on the color of the tip.

6. A device according to any one of claims 1 to 5, wherein the tip detection assembly is configured to detect differences in opacity of the at least a portion of the tip that is configured to be within the light path of the tip detection assembly when the tip is secured to the handpiece.

7. A device according to any one of claims 1 to 6, wherein the tip detection assembly is configured to detect differences in color or another property associated with light of the at least a portion of the tip that is configured to be within the light path of the tip detection assembly when the tip is secured to the handpiece.

8. The device of claim 7, wherein the color or another property determined the type of abrasive structure included on the tip.

9. A device according to any one of claims 1 to 8, wherein the at least a portion of the tip configured to be within the light path of the tip detection assembly when the tip is secured to the handpiece comprises a stem of the tip.

10. A device according to any one of claims 1 to 9, wherein the at least a portion of the tip configured to be within the light path of the tip detection assembly when the tip is secured to the handpiece comprises a dissolvable coating or layer.

11. The device of claim 10, wherein the dissolvable coating or layer comprises an opacity greater than 0%.

12. The device of claim 10 or 11, wherein the dissolvable coating or layer is provided along a fluid path of the tip.

13. A device according to any of claims 10 to 12, wherein the dissolvable coating or layer is configured to at least partially dissolve when fluid passes from the handpiece to the tip during use.

14. The device of claim 13, wherein the tip detection assembly is configured to detect an opacity or other light property of the at least a portion of the tip configured to be within the light path of the tip detection system when the tip is secured to the handpiece.

15. The device of claim 14, wherein the device is configured to terminate or not commence a skin treatment procedure if the opacity or other light property of the at least a portion of the tip configured to be within the light path of the tip detection system when the tip is secured to the handpiece is below a threshold level.

16. A device according to any of claims 10 to 15, wherein the device is configured to provide at least one alert if the opacity or other light property of the at least a portion of the tip configured to be within the light path of the tip detection system when the tip is secured to the handpiece is below a threshold level.

17. The device of claim 16, wherein the at least one alert comprises at least one of an audible alert, a visual alert and a haptic alert.

18. A device according to any one of claims 1 to 17, wherein the tip detection system comprises at least one light pipe or other light channel configured to transmit light from the at least one light emitter to the at least one light detector.

19. A device according to any one of claims 1 to 17, wherein the tip detection system does not comprise at least one light pipe or other light channel configured to transmit light from the at least one light emitter to the at least one light detector.

20. The device of claim 20, wherein a direct line of sight does not exist between the at least one light emitter and the at least one light detector.

21. A device according to any one of claims 1 to 17, wherein the at least one light emitter is adjacent to the at least one light detector.

22. The device of claim 21, wherein a direct line of sight exists between the at least one light emitter and the at least one light detector.

23. A method of controlling a skin treatment procedure, the method comprising:
detecting a property of a tip secured to a handpiece of a skin treatment device according to any one of claims 1 to 22; and
determining whether the skin treatment procedure should proceed or commence.

24. The method of claim 23, further comprising terminating the skin treatment procedure.

25. The method of claim 23 or 24, further comprising providing an alert.

26. The method of claim 25, wherein the alert comprises at least one of an audible alert, a visual alert and a haptic alert.

27. The method of claim 25, wherein the alert comprises sending an electronic communication.

28. A tip for use in a skin treatment device, the tip comprising:
a proximal portion configured to secure to a distal end of a handpiece of the skin treatment device, wherein the tip is configured to be placed in fluid communication with a fluid source when the tip is secured to the handpiece;
a distal portion configured to contact a target skin surface during use;
a fluid pathway placing the fluid source in fluid communication with the distal portion when the tip is secured to the handpiece; and
at least one detectable portion configured to be within a light path of a tip detection assembly of the handpiece when the tip is secured to the handpiece
wherein the handpiece is configured to detect a property of at least one detectable portion of the tip when the tip is secured to the handpiece.

29. The tip of claim 28, wherein the at least one detectable portion of the tip is positioned along an interior of a stem of the tip.

30. The tip of claim 28 or 29, wherein the tip is disposable.

31. A handpiece for use in skin treatment procedure, the handpiece comprising:
a main body;
a tip positioned along a distal end of the main body;
at least one fluid conduit configured to fluidly connect the tip to a fluid source;
at least one valve along the at least one fluid conduit, the at least one valve configured to regulate a flow of treatment fluid passing through the at least one fluid conduit; and
at least one controller configured to adjust a position of the at least one valve to regulate a flowrate of treatment fluid transferred to the tip, wherein the at least one controller comprises at least one electronic component to facilitate a communication between the at least one controller and the at least one valve.

32. The handpiece of claim 31, wherein the at least one valve comprises an electronically controlled valve.

33. The handpiece of claim 31 or 32, wherein the at least one valve comprises at least one of: a pinch valve, a needle valve, a stepper motor with a valve, a solenoid proportional valve, a piezoelectric valve or any other valve or flow control device.

34. A handpiece according to any one of the preceding claims, wherein the at least one controller comprises one or more buttons, switches, dials, knobs or any other component positioned along an exterior of the handpiece.

35. A handpiece according to any one of claims 31 to 33, wherein the at least one controller is not located on or in the handpiece.

36. The handpiece of claim 35, wherein the at least one controller is positioned on a separate system or computing device or component.

37. A handpiece according to any one of claims 31 to 36, further comprising at least one flowmeter configured to measure a flowrate of treatment fluid passing through the at least one fluid conduit.

38. The handpiece of claim 37, wherein the at least one flowmeter comprises a mechanical flowmeter.

39. The handpiece of claim 37, wherein the at least one flowmeter comprises an ultrasonic flowmeter.

40. A handpiece according to any one of claims 31 to 39, further comprising a force detection assembly, said force detection assembly being configured to detect a force or a pressure exerted by the tip on a subject's skin surface during use.

41. The handpiece of claim 40, wherein the force detection assembly comprises at least one transducer or other pressure sensor.

42. The handpiece of claims 40 or 41, further comprising at least one output that is configured to provide an indication of the force or pressure exerted by the tip on a subject's skin surface.

43. The handpiece of claim 42, wherein the at least one output is positioned on the handpiece.

44. The handpiece of claim 42, wherein the at least one output is not positioned on the handpiece.

45. The handpiece of claim 44, wherein the at least one output is located on a separate device or system.

46. A handpiece according to any one of claims 42 to 45, wherein the at least one output comprises a measured or estimated force or pressure.

47. A handpiece according to any one of claims 42 to 45, wherein the at least one output comprises a relative representation of a measured or estimated force or pressure.

48. The handpiece of claim 47, wherein the relative representation comprises an indication of “Low,” “Medium,” and “High.”

49. A handpiece according to any one of claims 1 to 48, wherein the tip is removably secured to the main body.

50. A handpiece according to any one of claims 1 to 49, further comprising a tip detection assembly configured to automatically detect a type of tip that is secured to the handpiece.

51. The handpiece of claim 50, wherein the tip detection assembly is configured to detect differences in color or other property associated with light.

52. A handpiece according to any one of claims 1 to 51, further comprising a container identification detection assembly configured to automatically detect a vial or other container positioned within or near the handpiece.

53. The handpiece of claim 52, wherein the container identification detection assembly comprises a RFID reader that is configured to read information from a RFID tag positioned on a vial or other container.

54. The handpiece of claim 52 or 53, wherein the container identification detection assembly is configured to automatically detect a vial or other container, regardless of an orientation of the vial or container within or near the handpiece.

55. A handpiece according to any one of claims 1 to 55, further comprising a light module, the light module positioned at or near a distal portion of the handpiece and configured to deliver light toward a subject's skin surface.

56. The handpiece of claim 55, wherein at least one property of light being emitted from the light module can be modified.

57. The handpiece of claim 56, wherein the at least one property comprises color, wavelength, intensity, brightness, duration and/or the like.

58. A handpiece according to any one of claims 55 to 57, wherein the light module further comprises at least one camera or at least one sensor.

59. The handpiece of claim 58, wherein the at least one camera or the at least one sensor is configured to receive data regarding a subject's skin surface.

60. A handpiece according to any one of the preceding claims, further comprising a temperature detection module or assembly, wherein the temperature detection module or assembly is positioned along a distal end of the handpiece and is configured to detect a temperature.

61. The handpiece of claim 60, wherein the temperature detection module or assembly is configured to detect a temperature of a skin surface being treated.

62. The handpiece of claim 60, wherein the temperature detection module or assembly is configured to detect a temperature of the treatment fluid.

63. A handpiece for use in skin treatment procedure, the handpiece comprising:
a main body;
a tip positioned along a distal end of the main body;
at least one fluid conduit configured to fluidly connect the tip to a fluid source;
at least one valve along the at least one fluid conduit, the at least one valve configured to regulate a flow of treatment fluid passing through the at least one fluid conduit; and
at least one flowmeter configured to measure a flowrate of treatment fluid passing through the at least one fluid conduit.

64. The handpiece of claim 63, wherein the at least one flowmeter comprises a mechanical flowmeter.

65. The handpiece of claim 63, wherein the at least one flowmeter comprises an ultrasonic flowmeter.

66. A handpiece according to any one of claims 63 to 65, wherein the at least one flowmeter is located upstream of the at least one valve.

67. A handpiece according to any one of claims 63 to 65, wherein the at least one flowmeter is located downstream of the at least one valve.

68. A handpiece for use in skin treatment procedure, the handpiece comprising:
a main body;
a tip positioned along a distal end of the main body; and
a force detection assembly, said force detection assembly being configured to detect a force or a pressure exerted by the tip on a subject's skin surface during use.

69. The handpiece of claim 68, wherein the force detection assembly comprises at least one transducer or other pressure sensor.

70. The handpiece of claims 68 or 69, further comprising at least one output that is configured to provide an indication of the force or pressure exerted by the tip on a subject's skin surface.

71. The handpiece of claim 70, wherein the at least one output is positioned on the handpiece.

72. The handpiece of claim 70, wherein the at least one output is not positioned on the handpiece.

73. The handpiece of claim 72, wherein the at least one output is located on a separate device or system.

74. A handpiece according to any one of claims 70 to 73, wherein the at least one output comprises a measured or estimated force or pressure.

75. A handpiece according to any one of claims 70 to 73, wherein the at least one output comprises a relative representation of a measured or estimated force or pressure.

76. The handpiece of claim 75, wherein the relative representation comprises an indication of “Low,” “Medium,” and “High.”

77. A handpiece for use in skin treatment procedure, the handpiece comprising:
a main body;
a tip positioned along a distal end of the main body; and
a container identification detection assembly configured to automatically detect a vial or other container positioned within or near the handpiece.

78. The handpiece of claim 77, wherein the container identification detection assembly comprises a RFID reader that is configured to read information from a RFID tag positioned on a vial or other container.

79. The handpiece of claim 77 or 78, wherein the container identification detection assembly is configured to automatically detect a vial or other container, regardless of an orientation of the vial or container within or near the handpiece.

80. A handpiece for use in skin treatment procedure, the handpiece comprising:
a main body;
a tip positioned along a distal end of the main body; and
a tip detection assembly configured to automatically detect a type of tip that is secured to the handpiece.

81. The handpiece of claim 80, wherein the tip detection assembly is configured to detect differences in color or other property associated with light.

82. A handpiece for use in skin treatment procedure, the handpiece comprising:
a main body;
a tip positioned along a distal end of the main body; and
a light module, the light module positioned at or near a distal portion of the handpiece and configured to deliver light toward a subject's skin surface.

83. The handpiece of claim 82, wherein at least one property of light being emitted from the light module can be modified.

84. The handpiece of claim 83, wherein the at least one property comprises color, wavelength, intensity, brightness, duration and/or the like.

85. A handpiece according to any one of claims 82 to 84, wherein the light module further comprises at least one camera or at least one sensor.

86. The handpiece of claim 85, wherein the at least one camera or the at least one sensor is configured to receive data regarding a subject's skin surface.

87. A handpiece for use in skin treatment procedure, the handpiece comprising:
a main body;
a tip positioned along a distal end of the main body; and
a temperature detection module or assembly, wherein the temperature detection module or assembly is positioned along a distal end of the handpiece and is configured to detect a temperature.

88. The handpiece of claim 87, wherein the temperature detection module or assembly is configured to detect a temperature of a treatment fluid being delivered to a subject via the handpiece.

89. The handpiece of claim 87 or 88, wherein the temperature detection module or assembly is configured to detect a temperature of skin surface being treated.

90. A method for treating skin, the method comprising:
delivering a treatment fluid to a tip of a handpiece, the handpiece comprising a main body and the tip, wherein the tip is positioned at or near a distal end of the main body;
wherein the handpiece comprises at least one fluid conduit configured to fluidly connect the tip to a fluid source; and
controlling at least one operational aspect of the handpiece using at least one controller of the handpiece, wherein the at least one controller comprises at least one electronic component to facilitate a communication between the at least one controller and at least one component of the handpiece.

91. The method of claim 90, wherein the at least one operational aspect comprises a flowrate of the treatment fluid to the tip.

92. The method of claim 90 or 91, wherein the at least one controller comprises one or more buttons, switches, dials, knobs or any other component positioned along an exterior of the handpiece.

93. A method according to any one of claims 90 to 92, wherein the at least one controller is not located on or in the handpiece.

94. The handpiece of claim 93, wherein the at least one controller is positioned on a separate system or computing device or component.

95. A method for treating skin, the method comprising:
delivering a treatment fluid to a tip of a handpiece, the handpiece comprising a main body and the tip, wherein the tip is positioned at or near a distal end of the main body;
wherein the handpiece comprises at least one fluid conduit configured to fluidly connect the tip to a fluid source; and
measuring or approximating a flowrate of treatment fluid passing through the at least one fluid conduit using a flowmeter.

96. The method of claim 95, further comprising regulating a flow of treatment fluid passing through the at least one fluid conduit.

97. The method of claim 96, wherein the handpiece comprises at least one valve along the at least one fluid conduit, the at least one valve configured to modify a flow of treatment fluid passing through the at least one fluid conduit.

98. A method according to any one of claims 95 to 97, wherein the at least one flowmeter comprises a mechanical flowmeter.

99. A method according to any one of claims 95 to 97, wherein the at least one flowmeter comprises an ultrasonic flowmeter.

100. A method for treating skin, the method comprising:
performing a skin treatment procedure using a tip of a handpiece, the handpiece comprising a main body and the tip, wherein the tip is positioned at or near a distal end of the main body; and
measuring or approximating a force or a pressure exerted by the tip on a subject's skin surface during use, wherein said measuring or approximating a force or a pressure is performed using a force detection assembly of the handpiece.

101. The method of claim 100, wherein the force detection assembly is positioned at least partially on or in the handpiece.

102. The method of claim 100 or 101, wherein the force detection assembly comprises at least one transducer or other pressure sensor.

103. A method according to any one of claims 100 to 102, further comprising providing an indication of the force or pressure exerted by the tip on a subject's skin surface using at least one output.

104. The method of claim 103, wherein the at least one output is provided on the handpiece.

105. The method of claim 103, wherein the at least one output is not provided on the handpiece.

106. The method of claim 105, wherein the at least one output is located on a separate device or system.

107. A method according to any one of claims 103 to 106, wherein the at least one output comprises a measured or estimated force or pressure.

108. A method according to any one of claims 103 to 107, wherein the at least one output comprises a relative representation of a measured or estimated force or pressure.

109. The method of claim 108, wherein the relative representation comprises an indication of “Low,” “Medium,” and “High.”

110. A method for treating skin, the method comprising:
performing a skin treatment procedure using a tip of a handpiece, the handpiece comprising a main body and the tip, wherein the tip is positioned at or near a distal end of the main body; and
automatically detecting a vial or other container positioned within or near the handpiece using a container identification detection assembly of the handpiece.

111. The method of claim 110, wherein the container identification detection assembly comprises a RFID reader that is configured to read information from a RFID tag positioned on a vial or other container.

112. The method of claim 110 or 111, wherein the container identification detection assembly is configured to automatically detect a vial or other container, regardless of an orientation of the vial or container within or near the handpiece.

113. A method for treating skin, the method comprising:
performing a skin treatment procedure using a tip of a handpiece, the handpiece comprising a main body and the tip, wherein the tip is positioned at or near a distal end of the main body; and
automatically detecting a type of tip that is secured to the handpiece using a detection assembly of the handpiece.

114. The method of claim 113, wherein the tip detection assembly is configured to detect differences in color or other property associated with light.

115. A method for treating skin, the method comprising:
performing a skin treatment procedure using a tip of a handpiece, the handpiece comprising a main body and the tip, wherein the tip is positioned at or near a distal end of the main body; and
selectively delivering light toward a subject's skin surface using a light module, the light module positioned at or near a distal portion of the handpiece.

116. The method of claim 115, further comprising modifying at least one property of light being emitted from the light module.

117. The method of claim 116, wherein the at least one property comprises color, wavelength, intensity, brightness, duration and/or the like.

118. A method according to any one of claims 115 to 117, wherein the light module further comprises at least one camera or at least one sensor.

119. The method of claim 118, wherein the at least one camera or the at least one sensor is configured to receive data regarding a subject's skin surface.

120. A method for treating skin, the method comprising:
performing a skin treatment procedure using a tip of a handpiece, the handpiece comprising a main body and the tip, wherein the tip is positioned at or near a distal end of the main body; and
measuring or approximating a temperature at, along or near a distal end of the handpiece using a temperature detection module or assembly, wherein at least one component of the temperature detection module or assembly is located at, along or near the tip.

121. A method for treating skin, the method comprising:
performing a skin treatment procedure using a tip of a handpiece, the handpiece comprising a main body and the tip, wherein the tip is positioned at or near a distal end of the main body;
delivering a treatment fluid to the tip using at least one fluid delivery conduit, wherein at least one valve in fluid communication with the at least one fluid delivery conduit is configured to regulate a flow of treatment fluid passing through the at least one valve;
generating a vacuum or positive pressure using a vacuum or positive pressure source fluidly coupled to the tip; and
controlling (a) a position of the at least one valve, and (b) at least one operational aspect of the vacuum or positive pressure using at least one controller, wherein the at least one controller comprises an electronic controller.

122. The method of claim 121, wherein the at least one controller comprises a single controller.

123. The method of claim 121, wherein the at least one controller comprises a first controller for controlling the position of the at least one valve, and a second controller for controlling at least one operational aspect of the vacuum or positive pressure.

124. A method according to any one of claims 121 to 123, wherein the handpiece comprises or is operatively coupled to a processor, the processor configured to help control the position of the at least one valve and/or the at least one operational aspect of the vacuum or positive pressure.

125. The method of claim 124, wherein the processor is configured to help control the position of the at least one valve and/or the at least one operational aspect of the vacuum or positive pressure based on a closed-loop or open-loop configuration or scheme.

126. A handpiece for use in a skin treatment procedure, the handpiece comprising:
a main body;
a tip positioned along a distal end of the main body;
at least one fluid conduit configured to fluidly connect the tip to a fluid source; and
at least one controller configured to adjust at least one parameter associated with an operation of the handpiece;
wherein the at least one controller comprises at least one electronic component.

127. The handpiece of claim 126, wherein the at least one controller comprises one or more buttons, switches, dials, knobs or any other component positioned along an exterior of the handpiece.

128. The handpiece of claim 126, wherein the at least one controller is controlled by manipulation, by audible command and/or by gesture control.

129. The handpiece according to any one of claims 126 to 128, wherein the at least one parameter associated with an operation of the handpiece comprises a flowrate of fluid passing through the at least one fluid conduit.

130. The handpiece according to any one of claims 126 to 128, wherein the at least one parameter associated with an operation of the handpiece comprises a selection of the fluid source to be delivered to the handpiece.

131. The handpiece according to any one of claims 126 to 128, wherein the at least one parameter associated with an operation of the handpiece comprises a selection of a vacuum force delivered to the tip during use.

132. The handpiece according to any one of claims 126 to 128, wherein the at least one parameter associated with an operation of the handpiece comprises regulation of an aspect of at least one separate modality.

133. The handpiece of claim 132, wherein the at least one separate modality comprises at least one of a light delivery element, an imaging element, a temperature modification element and/or the like.
